(19) United States
(12) Patent Application Publication
(10) Pub. No.: US 2007/0088358 A1
(43) Pub. Date: Apr. 19, 2007

(54) Minimally Invasive Spine Restoration Systems, Devices, Methods and Kits

(76) Inventors: Hansen Yuan, Fayetteville, NY (US); Mark K. Kuiper, Seattle, WA (US); Anthony V. Finazzo, Lake Forest Park, WA (US); John Arthur Ohrt, Redmond, WA (US); Cin Abidin, Issaquah, WA (US); Mark A. Reiley, Piedmont, CA (US); David Stinson, Woodinville, WA (US); Lawrence R. Jones, Conifer, CO (US); Sean Sung-Ho Suh, Kirkland, WA (US); Richard J. Broman, Kirkland, WA (US); Michael J. Funk, North Bend, WA (US); Phillip Berg, Federal Way, WA (US); Martha K. Stone, Lake Forest Park, WA (US); Matthew Quest, Bothell, WA (US)

Correspondence Address: WILSON SONSINI GOODRICH & ROSATI, 650 PAGE MILL ROAD, PALO ALTO, CA, 94304-1050, US

(21) Appl. No.: 11/277,223

(22) Filed: Mar. 22, 2006

Related U.S. Application Data
(60) Provisional application No. 60/664,441, filed on Mar. 22, 2005.
(60) Provisional application No. 60/719,427, filed on Sep. 22, 2005.
(60) Provisional application No. 60/752,277, filed on Dec. 20, 2005.

(51) Int. Cl. A61F 2/30

(52) U.S. Cl. 606/61

(57) ABSTRACT

The invention discloses methods and devices for repairing, replacing and/or augmenting natural facet joint surfaces and/or facet capsules. A facet joint restoration device of the invention for use in a restoring a facet joint surface comprises: a cephalad facet joint element comprising a flexible member adapted to engage a first vertebrae and an artificial cephalad joint; and a caudad facet joint element comprising a connector adapted for fixation to a second vertebrae and an artificial caudad joint adapted to engage the cephalad facet joint. In another embodiment, the invention discloses a facet joint replacement device for use in replacing all or a portion of a natural facet joint between a first vertebrae and a second vertebrae comprising: a first cephalad facet joint element having a fixation member adapted to engage a lamina or spinous process of the first vertebrae and a first caudad facet joint element, the first caudad facet joint element comprising a first caudad connector adapted to fixate to the second vertebral body and an artificial caudad facet surface adapted to engage with the cephalad facet joint element.

### CROSS-REFERENCE

This application claims the benefit of U.S. Provisional Application No. 60/664,441, to Michael J. Funk et al, filed Mar. 22, 2005, and entitled “Minimally Invasive Facet Replacement”; U.S. Provisional Application No. 60/719,427, to Michael J. Funk et al., filed Sep. 22, 2005, entitled “Prosthesis, Tools and Methods for Replacement of Natural Facet Joints with Artificial Facet Joint Surfaces”; and U.S. Provisional Application 60/752,277 to Christopher Ralph et al., filed Dec. 20, 2005, entitled “Spinal Joint Replacement Systems”; the disclosures of which are incorporated herein by reference in their entireties.

### FIELD OF THE INVENTION

The present invention generally relates to devices and surgical methods for the treatment of various types of pathologies of the spine. More specifically, the present invention is directed to several different types of minimally invasive devices, methods, systems and kits for treating injured or diseased facet joints, intervertebral Joints and adjacent anatomy of the spine.

### BACKGROUND OF THE INVENTION

Back pain, particularly in the “small of the back” or lumbosacral (L4-S1) region, shown in FIG. 1, is a common ailment. In many cases, the pain severely limits a person's functional ability and quality of life. Such pain can result from a variety of spinal pathologies. Through disease or injury, the laminae, spinous process, articular processes, or facets of one or more vertebral bodies can become damaged, such that the vertebrae no longer articulate or properly align with each other. This can result in an undesired anatomy, loss of mobility, and pain or discomfort.

In many cases, the vertebral facet joints can be damaged by either traumatic injury or by various disease processes. These disease processes include osteoarthritis, ankylosing spondylolysis, and degenerative spondylolisthesis. Moreover, the facet joint has been implicated as a potential cause of neck pain for persons having whiplash. Aside from pain coming from the facets themselves, such damage to the facet joints can often result in eventual degeneration, abrasion, or wearing down of the facet joints, eventually resulting in pressure on nerves, also called “pinched” nerves, or nerve compression or impingement. The result is further pain, misaligned anatomy, and a corresponding loss of mobility. Pressure on nerves can also occur without an anatomic or functional manifestation of a disease, or pathology, at the facet joint, e.g., as a result of a herniated disc.

Many spinal pathologies mandating repair and/or replacement of an intervertebral disc (including many of those that may be currently treated through spinal fusion, interspinous distraction and/or dynamic stabilization), can often be traced back to degeneration, disease and/or failure of the facet joints. Alteration of the facet joint biomechanics resulting from an anatomic or functional manifestation of a disease can adversely affect the loading and biomechanics of the intervertebral disc, eventually resulting in degeneration, damage and/or failure of the intervertebral disc.

One type of conventional treatment of facet joint pathology is spinal stabilization, also known as intervertebral stabilization. Intervertebral stabilization desirably prevents relative motion between vertebrae of the spine. By preventing movement, pain can be reduced. Stabilization can be accomplished by various methods. One method of stabilization is spinal fusion. Another method of stabilization is fixation of any number of vertebrae to stabilize and prevent movement of the vertebrae. In addition, where compression or subsidence of the disc and/or facet joints has occurred, the physician can utilize fusion devices such as pedicle screw and rods systems, or interbody fusion cages, to elevate or “jack up” the compressed level, desirably obtaining a more normal anatomical spacing between the vertebral bodies.

Various devices are known for fixing the spine and/or sacral bone adjacent the vertebra, as well as attaching devices used for fixation, are known in the art including: U.S. Pat. Nos. 6,290,703, to Ganem, for Device for Fixing the Sacral Bone to Adjacent Vertebrae During Osteosynthesis of the Backbone; U.S. Pat. No. 6,547,790, to Harkey, III, et al., for Orthopaedic Rod/Plate Locking Mechanisms and Surgical Methods; U.S. Pat. No. 6,074,391, to Metz-Stavenhagen, et al., for Receiving Part for a Retaining Component of a Vertebral Column Implant; U.S. Pat. No. 5,569,247, to Morrison, for Enhanced Variable Angle Bone Bolt; U.S. Pat. No. 5,891,145, to Morrison, et al., for Multi-Axial Screw; U.S. Pat. No. 6,090,111, to Nichols, for Device for Securing Spinal Rods; U.S. Pat. No. 6,451,021, to Ralph, et al., for Polyaxial Pedicle Screw Having a Rotating Locking Element; U.S. Pat. No. 5,683,392, to Richelsoph, et al., for Multi-Planar Locking Mechanism for Bone Fixation; U.S. Pat. No. 5,863,293, to Richelsoph, for Spinal Implant Fixation Assembly; U.S. Pat. No. 5,964,760, to Richelsoph, for Spinal Implant Fixation Assembly; U.S. Pat. No. 6,010,503, to Richelsoph, et al., for Locking Mechanism; U.S. Pat. No. 6,019,759, to Rogozinski, for Multi-Directional Fasteners or Attachment Devices for Spinal Implant Elements; U.S. Pat. No. 6,540,749, to Schafer, et al., for Bone Screw; U.S. Pat. No. 6,077,262, to Schlapfer, for Posterior Spinal Implant; U.S. Pat. No. 6,248,105, to Schlapfer, et al., for Device for Connecting a Longitudinal Support with a Pedicle Screw; U.S. Pat. No. 6,524,315, to Selvitelli, et al., for Orthopaedic Rod/Plate Locking Mechanism; U.S. Pat. No. 5,797,911, to Sherman, et al., for Multi-Axial Bone Screw Assembly; U.S. Pat. No. 5,879,350, to Sherman, et al., for Multi-Axial Bone Screw Assembly; U.S. Pat. No. 5,885,285, to Simonson, For Spinal Implant Connection Assembly; U.S. Pat. No. 5,643,263, to Simonson for Spinal Implant Connection Assembly; U.S. Pat. No. 6,565,565, to Yuan, et al., for Device for Securing Spinal Rods; U.S. Pat. No. 5,725,527, to Biederman, et al., for Anchoring Member; U.S. Pat. No. 6,471,705, to Biederman, et al., for Bone Screw; U.S. Pat. No. 5,575,792, to Errico, et al., for Extending Hook and Polyaxial Coupling Element Device for Use with Top Loading Rod Fixation Devices; U.S. Pat. No. 5,688,274, to Errico, et al., for Spinal Implant Device having a Single Central Rod and Claw Hooks; U.S. Pat. No. 5,690,630, to Errico, et al., for Polyaxial Pedicle Screw; U.S. Pat. No. 6,022,350, to Ganem, for Bone Fixing Device, in Particular for Fixing to the Sacmum during Osteosynthesis of the Backbone; U.S. Pat. No. 4,805,602, to Puno, et al., for Transpedicular Screw and Rod System; U.S. Pat. No. 5,474,555, to Puno, et al., for Spinal Implant System; U.S. Pat. No. 4,611,581, to Steffee, for Apparatus for Straightening Spinal Columns; U.S. Pat. No. U.S. Pat. No. 5,129,900, to Asher, et al., for Spinal Column Retaining Method and Apparatus; U.S. Pat. No. 5,741,255, to Krag, et al., for Spinal Column Retaining Apparatus; U.S. Pat. No. 6,132,430, to Wagner, for Spinal Fixation System; U.S. Publication No. 2002/0120272, and to Yuan, et al., for Device for Securing Spinal Rods.

Another type of conventional spinal treatment is decompressive laminectomy. Where spinal stenosis (or other spinal pathology) results in a narrowing of the spinal canal and/or the intervertebral foramen (through which the spinal nerves exit the spine), and neural impingement, compression and/or pain results, the tissue(s) (hard and/or soft tissues) causing the narrowing may need to be resected and/or removed. A procedure which involves excision of part or all of the laminae and other tissues to relieve compression of nerves is called a decompressive laminectomy. See, for example, U.S. Pat. Nos. 5,019,081, to Watanabe, for Laminectomy Surgical Process; U.S. Pat. No. 5,000,165, to Watanabe, for Lumbar Spine Rod Fixation System; and U.S. Pat. No. 4,210,317, to Spann, et al., for Apparatus for Supporting and Positioning the Arm and Shoulder. Depending upon the extent of the decompression, the removal of support structures such as the facet joints and/or connective tissues (either because these tissues are connected to removed structures or are resected to access the surgical site) may result in instability of the spine, necessitating some form of supplemental support such as spinal fusion, discussed above.

### SUMMARY OF THE INVENTION

While spinal fusion has become the “gold standard” for treating many spinal pathologies, including pathologies such as neurological involvement, intractable pain, instability of the spine and/or disc degeneration, it would be desirable to reduce and/or obviate the need for spinal fusion procedures by providing devices and systems that stabilize, or preserve motion of the spinal motion segment (including, but not limited to, facet joint repair or replacement, intervertebral disk replacement or nucleus replacement, implantation of interspinous spacers and/or dynamic stabilization devices, and/or facet injections).

The present invention includes the recognition that many spinal pathologies eventually requiring surgical intervention can be traced back, in their earlier stage(s), to some manner of a degeneration, disease and/or failure of the facet joints. Moreover, spinal fusion procedures can eventually require further surgical intervention. For example, degeneration of facet joints can result in an unnatural loading of an intervertebral disc, eventually resulting in damage to the disc, including annular bulges and/or tears. Similarly, degeneration and/or failure of a facet joint can potentially lead to slipping of the vertebral bodies relative to one another, potentially resulting in spondylolisthesis and/or compression of nerve fibers. In addition, degeneration of the facet joints themselves can become extremely painful, leading to additional interventional procedures such as facet injections, nerve blocks, facet removal, facet replacement, and/or spinal fusion. Thus, if the degenerating facet joint can be treated at an early stage, the need for additional, more intrusive procedures, may be obviated and damage that has already occurred to spinal structures such as the intervertebral disc of the treated level (as well as the disc and/or facets of other spinal levels) may be slowed, halted or even reversed.

Further, the invention includes the ability to accommodate anatomical variability to treat all vertebral levels, including L3-L4, L4-L5 and L5-S1, across a majority of the patient population.

The various embodiments disclosed and discussed herein may be utilized to restore and/or maintain varying levels of the quality or state of motion or mobility and/or motion preservation in the treated vertebral bodies. Depending upon the extent of facet joint degradation, and the chosen treatment regime(s), it may be possible to completely restore the quality or state of motion across the entire spinal motion segment, across one or more of the facet joints, or restore limited motion across the facet joint(s) to reduce or obviate the need for further treatment of the spinal motion segment.

A facet joint restoration device for use in a restoring a facet joint surface comprising: a cephalad facet joint element comprising (1) a flexible member adapted to engage a first vertebrae and (2) an artificial cephalad joint; and a caudad facet joint element comprising (1) a connector adapted for fixation to a second vertebrae and (2) an artificial caudad joint adapted to engage the cephalad facet joint. In some embodiments, the flexible member is adapted to engage a lamina of the first vertebrae. In other embodiments, the cephalad facet joint further comprises a plate with an anchoring mechanism adapted to engage a lamina of the first vertebrae. The anchoring mechanisms can be any suitable mechanism, including, for example, one or more anchoring mechanisms selected from the group consisting of teeth, ridges, nubs, serrations, granulations, a stem, a screw and spikes. In some embodiments, the cephalad facet joint element can be further adapted to comprises a second anchoring mechanism for securing the cephalad facet joint element to the first vertebrae. Further, the connector can be adapted to provide for fixation to a pedicle of the second vertebrae. In some embodiments it may be desirable for the second anchoring mechanism to further comprise a bony in-growth surface. As will be appreciated by those skilled in the art, in still other embodiments, the device can be configured to replace tissue removed from the facet joint, such as where the facet joint is resected. In still other embodiments, the device is adapted to restore or maintain motion or mobility for the facet joint. Further, a surface of one of the cephalad facet joint element or caudad facet joint element can be adapted to contour to an opposing mating surface. For example, the artificial caudad joint is a caudad cup having a concave surface. In some embodiments, the flexible member is a flexible cable. The flexible cable may be surrounded by a tube and/or may be further adapted to engage a lock. A spring washer adapted to engage a surface of the first or second vertebrae can be used in some embodiments, if desired. Further, it may be desirable to employ a malleable plate adapted to engage a laminar surface to support the cephalad facet joint element during implantation in other embodiments,

A facet joint replacement device for use in replacing all or a portion of a natural facet joint between a first vertebrae and a second vertebrae comprising: a first cephalad facet joint element having a fixation member adapted to engage a lamina or spinous process of the first vertebrae and a first caudad facet joint element, the first caudad facet joint element comprising a first caudad connector adapted to fixate to the second vertebral body and an artificial caudad facet surface adapted to engage with the cephalad facet joint element. In some embodiments, the fixation member is a flexible cable. A second cephalad facet joint element and a first crossbar can be adapted in some embodiments to connect the first cephalad facet joint element to the second cephalad facet joint element. In still other embodiments, a second caudad facet joint element and a first crossbar adapted to connect the first caudad facet joint element to the second caudad facet joint element. As will be appreciated by those skilled in the art, a second caudad facet joint element and a second crossbar adapted to connect the first caudad facet joint element to the second caudad facet joint element may be desirable in still other embodiments. The devices of the invention can further be adapted to include a laminar clamp. In some embodiments, it may be desirable for the laminar clamp to be adapted to engage the first cephalad facet joint element. In other embodiments, laminar clamp further comprises teeth for engaging a laminar surface. The laminar clamp can be further comprised of a first component and a second component adapted to adjustably engage the lamina. In some embodiments the first cephalad facet joint element is adapted to extend from the laminar clamp. Further, in other embodiments it may be desirable for the artificial caudad facet surface to be further adapted to comprise a caudad cup. In some instances, the first cephalad facet joint element can be adapted to rotatably engages the fixation member in some embodiments. In still other embodiments it may be desirable for the flexible cable to be surrounded by a tube. The facet replacement device of an embodiment can be further adapted to comprise a malleable plate adapted to engage a laminar surface to support the cephalad facet joint element.

A functional spine unit restoration system for use in a functional spine unit at a vertebal level in a spine comprising: a first and second cephalad facet joint element; a first and second caudad facet joint element comprising a connector adapted to secure a vertebral body and an artificial caudad joint adapted to engage the cephalad fact joint; a crossbar adapted to engage the first caudad facet joint element at a first end and the second caudad facet joint element at a second end; and an artificial intervertebral disc. In some embodiments of the invention, the anchor is a flexible cable. The cephalad facet joint can further comprise a plate with an anchoring mechanism adapted to engage the lamina. In still other embodiments the anchoring mechanism includes one or more anchoring mechanisms selected from the group consisting of teeth, ridges, nubs, serrations, granulations, a stem, and spikes. The plate can further be adapted to comprise a threaded rod adapted and configured to engage a threaded aperture of a bearing. The devices can also be configured from naturally occurring materials adapted to form the device, ceramic, metal, or polymer, or combinations thereof. In operation of the embodiments, the device restores the biomechanical operation of the functional spine unit. The device treats degenerating or diseased tissue in the target functional spine unit. In some embodiments, the device is adapted to restore or maintain motion or mobility for the target functional spine unit. In some instances a surface of one of the cephalad joint or caudad joint is adapted to contour to an opposing mating surface. In other instances, a surface of one of the cephalad joint or caudad joint is adapted to contour to an opposing mating surface. The flexible cable can be adapted in some embodiments to engage a lock. In still other embodiments, the system further comprises a laminar clamp. The laminar clamp can be adapted to engage the crossbar. Further, the laminar clamp can comprise teeth for engaging a laminar surface. In some embodiments, the laminar clamp is further comprised of a first component and a second component adapted to adjustably engage the lamina. The cephalad joints can be adapted to extend from the laminar clamp. Further, the laminar clamp can be adapted to engage the crossbar. In some embodiments, the orientation of a first cephalad joint to a first caudad joint is different than an orientation of a second cephalad joint to a second caudad joint. In still other embodiments, the laminar clamp is adjustable along a length parallel to a midline of the spine. As with other embodiments, the artificial caudad joint can be adapted to form a caudad cup. In still other embodiments, the artificial cephalad joint rotatably engages the flexible cable; the flexible cable can be surrounded by a tube. In some cases, a spring washer may be employed to engage a surface of a vertebral body. Further embodiments can be adapted to engage a malleable plate that engages a laminar surface to support the cephalad joint element during implantation.

A kit for restoring a functional spine unit at a vertebral level in a spine comprising: a first and second cephalad facet joint element; a first and second caudad facet joint element comprising a connector adapted to secure a vertebral body and an artificial caudad joint adapted to engage the cephalad fact joint; a crossbar adapted to engage the first caudad facet joint element at a first end and the second caudad facet joint element at a second end; and an artificial intervertebral disc.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a lateral elevation view of a normal human spinal column;

FIG. 2A is a superior view of a normal human lumbar vertebra;

FIG. 2B is a lateral elevational view of two vertebral bodies forming a functional spinal unit;

FIG. 2C is a posterior view of two vertebral bodies forming a functional spine unit and illustrating a coronal plane across a facet joint;

FIG. 2D is a cross-sectional view of a single facet joint in a spinal column taken along a coronal plane;

FIG. 2E is a posterolateral oblique view of a vertebrae from a human spinal column;

FIG. 3 is a perspective view of the anatomical planes of the human body;

FIG. 4 depicts an embodiment of a facet replacement device according to the invention;

FIG. 5 illustrates a bilateral facet replacement system according to the invention;

FIG. 6A illustrates two components of the facet replacement system;

FIGS. 6B-C illustrate the two components illustrated in FIG. 6A in combination from different perspectives;

FIGS. 7A-C illustrate an implanted facet replacement device according to the invention from a posterior and lateral perspective;

FIGS. 8A-D illustrate an implanted facet replacement device according to another embodiment of the invention from a posterior and lateral perspective;

FIGS. 9A-B illustrate a facet replacement device according to another embodiment of the invention from a side view and a top view;

FIGS. 10A-C illustrate the facet replacement device of FIGS. 9A-B implanted from a posterior and lateral view;

FIG. 11 illustrates a bilateral facet replacement system with a cross-bar;

FIGS. 12A-B illustrate a bilateral facet replacement system with a cross-bar according to another embodiment of the invention;

FIGS. 12C-D illustrates the facet replacement system implanted from different perspectives;

FIGS. 13A-B illustrate a facet replacement system having caudad cups, a cross-bar and a laminar clamp;

FIGS. 13C-D illustrate the clamp portion of the system implanted;

FIG. 14A illustrates a facet replacement system according to an alternate embodiment wherein the laminar clamp has teeth;

FIGS. 14B-D illustrate the facet replacement system of FIG. 14A implanted from posterior and lateral views;

FIGS. 15A-E illustrates a facet replacement system according to an alternate embodiment implanted from various perspectives;

FIG. 16A illustrates a facet replacement system according to an alternate embodiment wherein the laminar clamp has a modular clamp assembly;

FIGS. 16B-C illustrate the facet replacement system of FIG. 16A implanted from various perspectives;

FIGS. 17A-C illustrate a facet replacement system according to an alternate embodiment, and the system implanted from various perspectives;

FIG. 18A illustrates a facet replacement system according to an alternate embodiment wherein the laminar clamp is an adjustable c-clamp;

FIGS. 18B-C illustrate the facet replacement system of FIG. 18A implanted from various perspectives;

FIG. 19A illustrates a facet replacement system according to an alternate embodiment wherein the laminar clamp has adjustable rods;

FIGS. 19B-C illustrate the facet replacement system of FIG. 19A implanted from various perspectives;

FIG. 19D illustrates a facet replacement system according to an alternate embodiment wherein the laminar clamp has adjustable rods;

FIG. 19E illustrates the facet replacement system of FIG. 19D implanted;

FIG. 20A illustrates a facet replacement system according to an alternate embodiment wherein the laminar clamp has adjustable rods and at least one portion of the cross-arm is anchorable directly into the cephalad vertebrae;

FIGS. 20B-D illustrate the facet replacement system of FIG. 20A implanted from various perspectives;

FIG. 21A illustrates a facet replacement system according to an alternate embodiment wherein the laminar clamp has a linking mechanism;

FIGS. 21B-D illustrate the facet replacement system of FIG. 21A implanted from various perspectives;

FIG. 22A illustrates a facet replacement system according to an alternate embodiment wherein the laminar clamp has an anterior facing hook for engaging part of the vertebral body;

FIGS. 22B-D illustrate the facet replacement system of FIG. 22A implanted from various perspectives;

FIG. 23 is a side view of one side of a facet replacement system;

FIG. 24A is a perspective view of a cross-bar mount;

FIG. 24B-C illustrate the cross-bar mount of FIG. 24A implanted from a posterior view and a side view;

FIG. 25A is a top view of a cephalad interconnection device according to the invention;

FIG. 25B-D illustrate the cross-bar mount of FIG. 25A implanted from a posterior view, superior view and a lateral view;

FIG. 26 is a perspective view of a facet arthroplasty system particularly well-suited for use in conjunction with an artificial intervertebral disc replacement (not shown);

FIGS. 27A-H illustrate the components of a translaminar facet arthroplasty cephalad construct system;

FIGS. 28A-B illustrate the translaminar facet arthroplasty cephalad construct system showing its construction and operation;

FIGS. 29A-C illustrate the translaminar facet arthroplasty cephalad construct system according to an alternate embodiment showing its construction and operation;

FIGS. 30A-c illustrate the translaminar facet arthroplasty cephalad construct system according to an alternate embodiment showing its construction;

FIGS. 31A-B illustrate a plate suitable for use with the fixation bearing systems described herein;

FIGS. 32A-C illustrate cross-sections of an alternate cephalad bearing fixation system;

FIGS. 33A-F illustrate various views of a fixation device suitable for use at a sacral level;

FIGS. 34A-B illustrates a translaminar fixation system incorporating the use of a spring washer;

FIGS. 35A-B illustrates a disc replacement device with a facet replacement component; and

FIGS. 36A-B illustrates a disc replacement device according to an alternative embodiment with a facet replacement component.

### DETAILED DESCRIPTION OF THE INVENTION

The invention relates generally to implantable devices, apparatus or mechanisms that are suitable for implantation within a human body to restore, augment, and/or replace hard tissue, soft tissue and/or connective tissue, including bone and cartilage, and systems for treating the anatomic or functional manifestation of injury or diseases, such as spinal pathologies. In some instances, the implantable devices can include devices designed to replace missing, removed, or resected body parts or structure. The implantable devices, apparatus or mechanisms are configured such that the devices can be formed from parts, elements or components which alone or in combination comprise the device. The implantable devices can also be configured such that one or more elements or components are formed integrally to achieve a desired physiological, operational or functional result such that the components complete the device. Functional results can include the surgical restoration and functional power of a joint, controlling, limiting or altering the functional power of a joint, and/or eliminating the functional power of a joint by preventing joint motion. Portions of the device can be configured to replace or augment existing anatomy and/or implanted devices, and/or be used in combination with resection or removal of existing anatomical structure.

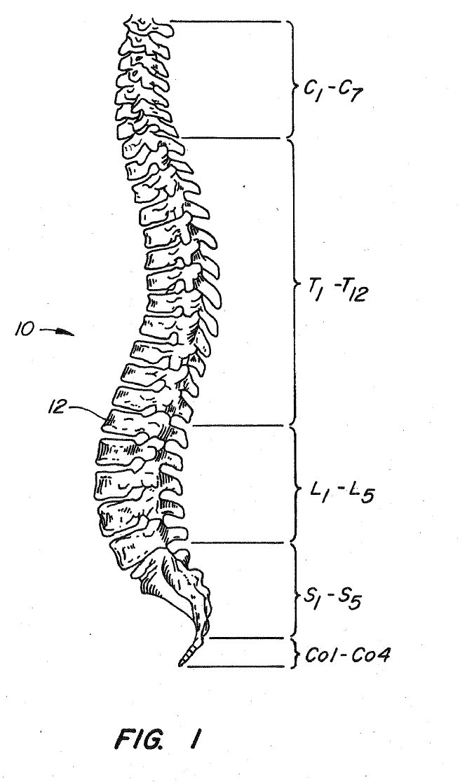

The devices of the invention are designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five normally-fused vertebrae, known as S1-S5, while the coccygeal region contains four fused vertebrae, known as Co1-Co4.

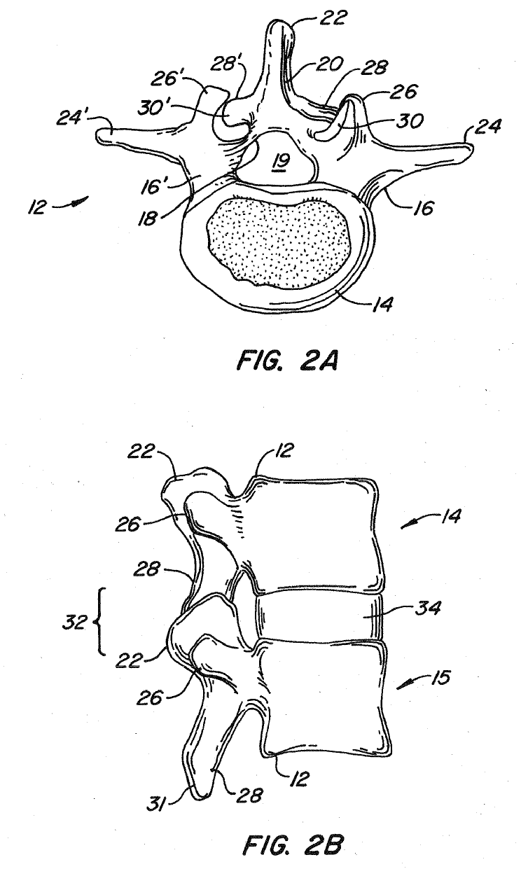

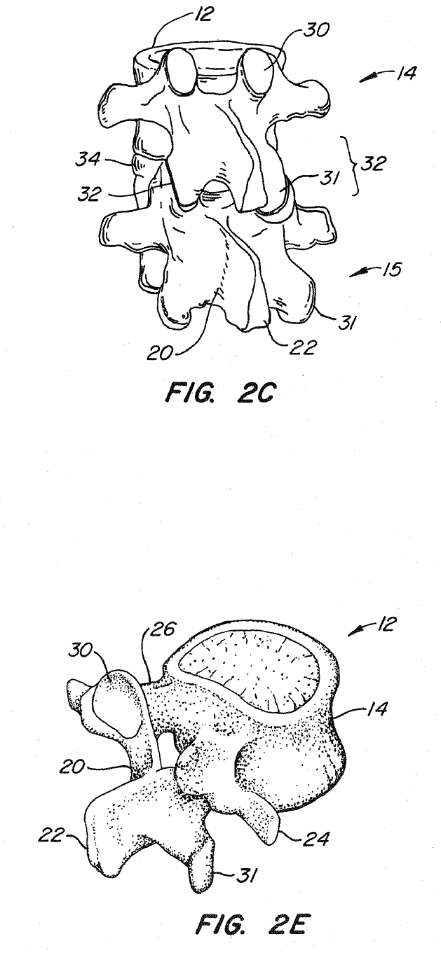

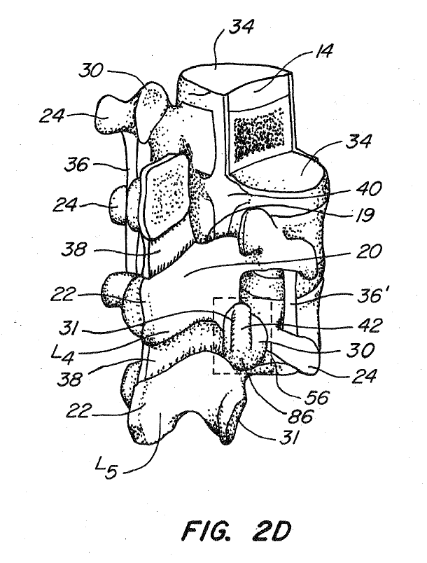

An example of one vertebra is illustrated in FIG. 2A which depicts a superior plan view of a normal human lumbar vertebra 12. Although human lumbar vertebrae vary somewhat according to location, the vertebrae share many common features. Each vertebra 12 includes a vertebral body 14. Two short bony protrusions, the pedicles 16, 16′, extend dorsally from each side of the vertebral body 14 to form a vertebral arch 18 which defines the vertebral foramen 19.

At the posterior end of each pedicle 16, the vertebral arch 18 flares out into broad plates of bone known as the laminae 20. The laminae 20 fuse with each other to form a spinous process 22. The spinous process 22 provides for muscle and ligamentous attachment. A smooth transition from the pedicles 16 to the laminae 20 is interrupted by the formation of a series of processes.

Two transverse processes 24, 24′ thrust out laterally, one on each side, from the junction of the pedicle 16 with the lamina 20. The transverse processes 24, 24′ serve as levers for the attachment of muscles to the vertebrae 12. Four articular processes, two superior 26, 26′ and two inferior 28, 28′, also rise from the junctions of the pedicles 16 and the laminae 20. The superior articular processes 26, 26′ are sharp oval plates of bone rising upward on each side of the vertebrae, while the inferior processes 28, 28′ are oval plates of bone that jut downward on each side. See also FIGS. 2B and 2D.

The superior and inferior articular processes 26 and 28 each have a natural bony structure known as a facet. The superior articular facet 30 faces medially upward, while the inferior articular facet 31 (see FIGS. 2B-E) faces laterally downward. When adjacent vertebrae 12 are aligned, the facets 30 and 31, capped with a smooth articular cartilage and encapsulated by ligaments, interlock to form a facet joint 32. The facet joints are apophyseal joints that have a loose capsule and a synovial lining.

As discussed above, the facet joint 32 is composed of a superior facet and an inferior facet. The superior facet is formed by the vertebral level below the joint 32, and the inferior facet is formed in the vertebral level above the joint 32. For example, in the L4-L5 facet joint shown in FIG. 2B, the superior facet of the joint 32 is formed by bony structure on the L5 vertebra (i.e., a superior articular surface and supporting bone 26 on the L5 vertebra), and the inferior facet of the joint 32 is formed by bony structure on the L4 vertebra (i.e., an inferior articular surface and supporting bone 28 on the L4 vertebra). The angle formed by a facet joint located between a superior facet and an inferior facet changes with respect to the midline of the spine depending upon the location of the vertebral body along the spine 10 (FIG. 1). The facet joints do not, in and of themselves, substantially support axial loads unless the spine is in an extension posture (lordosis). As would be appreciated by those of skill in the art, the orientation of the facet joint for a particular pair of vertebral bodies changes significantly from the thoracic to the lumbar spine to accommodate a joint's ability to resist flexion-extension, lateral bending, and rotation.

An intervertebral disc 34 between each adjacent vertebra 12 (with stacked vertebral bodies shown as 14, 15 in FIGS. 2B, C, E) permits gliding movement between the vertebrae 12. The structure and alignment of the vertebrae 12 thus permit a range of movement of the vertebrae 12 relative to each other. FIG. 2E illustrates a posterolateral oblique view of a vertebrae 12, further illustrating the curved surface of the superior articular facet 30 and the protruding structure of the inferior facet 31 adapted to mate with the opposing superior articular facet. As discussed above, the position of the inferior facet 31 and superior facet 30 varies on a particular vertebral body to achieve the desired biomechanical behavior of a region of the spine.

Thus, the overall spine comprises a series of functional spinal units that are a motion segment consisting of two adjacent vertebral bodies (e.g., 14, 15 of FIGS. 2 B, C, E), the intervertebral disc (e.g., 34 of FIGS. 2 B, C, E), associated ligaments, and facet joints (e.g., 32 of FIG. 2D). See, Posner, I, et al. A biomechanical analysis of the clinical stability of the lumbar and lumbrosacral spine. Spine 7:374-389 (1982).

As previously described, a natural facet joint, such as facet joint 32 (FIGS. 2B-D), has a superior facet 30 and an inferior facet 31 (shown in FIGS. 2 B, C, E). In anatomical terms, the superior facet of the joint is formed by the vertebral level below the joint, which can thus be called the “caudad” portion of the facet joint because it is anatomically closer to the tail bone or feet of the person. The inferior facet of the facet joint is formed by the vertebral level above the joint, which can be called the “cephalad” portion of the facet joint because it is anatomically closer to the head of the person. Thus, a device that, in use, replaces the caudad portion of a natural facet joint (i.e., the superior facet 30) can be referred to as a “caudad” device. Likewise, a device that, in use, replaces the cephalad portion of a natural facet joint (i.e., the inferior facet 31) can be referred to a “cephalad” device.

As will be appreciated by those skilled in the art, it can be difficult for a surgeon to determine the precise size and/or shape necessary for an implantable device until the surgical site has actually been prepared for receiving the device. In such case, the surgeon typically would desire to quickly deploy a family of devices and/or device components possessing differing sizes and/or shapes during the surgery. Thus, embodiments of the spinal devices of the present invention include modular designs that are either or both configurable and adaptable. Additionally, the various embodiments disclosed herein may also be formed into a “kit” or system of modular components that can be assembled in situ to create a patient specific solution. As will be appreciated by those of skill in the art, as imaging technology improves, and mechanisms for interpreting the images (e.g., software tools) improve, patient specific designs employing these concepts may be configured or manufactured prior to the surgery. Thus, it is within the scope of the invention to provide for patient specific devices with integrally formed components that are pre-configured.

The devices of the present invention are configurable such that the resulting implantable device is selected and positioned to conform to a specific anatomy or desired surgical outcome. The adaptable aspects of embodiments of the present invention provide the surgeon with customization options during the implantation or revision procedure. It is the adaptability of the present devices and systems that also provides adjustment of the components during the implantation procedure to ensure optimal conformity to the desired anatomical orientation or surgical outcome. An adaptable modular device of the present invention allows for the adjustment of various component-to-component relationships. One example of a component-to-component relationship is the rotational angular relationship between an anchoring device and the device to be anchored. Other examples of the adaptability of modular device of the present invention are as described in greater detail below. Configurability may be thought of as the selection of a particular size of component that together with other component size selections results in a “custom fit” implantable device. Adaptability then can refer to the implantation and adjustment of the individual components within a range of positions in such a way as to fine tune the “custom fit” devices for an individual patient. The net result is that embodiments of the modular, configurable, adaptable spinal device and systems of the present invention allow the surgeon to alter the size, orientation, and relationship between the various components of the device to fit the particular needs of a patient during the actual surgical procedure.

To prepare the anatomy for implantation of the devices and systems disclosed herein, it may be desirable to alter or remove anatomy from the patient. For example, common ligaments, such as capsular ligaments, anterior longitudinal ligaments, interspinous ligaments and/or ligamentum flavum may be altered or removed, as well as portions of the cephalad and/or caudad vertebra, including inferior/superior facets, or portions thereof. Alternatively, less-invasive and/or minimally-invasive surgical tools and techniques are provided that, among other things, limit the need for resection and/or alteration of such anatomy, which desirably allows for greater retention of natural anatomical features that can (1) stabilize the spine, thereby desirably reducing loads experienced by the facet replacement device, and/or (2) load-share with the facet joint replacement device in bearing physiological loads.

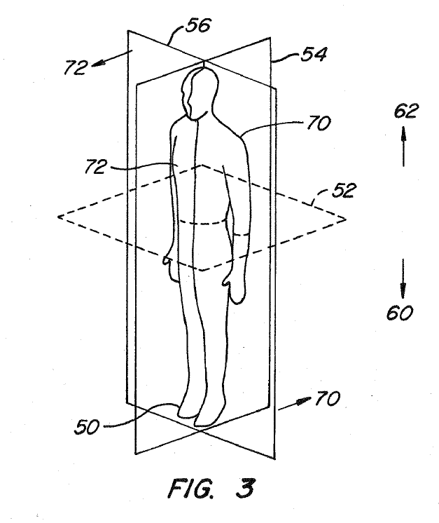

In order to understand the configurability, adaptability and operational aspects of the invention, it is helpful to understand the anatomical references of the body 50 with respect to which the position and operation of the devices, and components thereof, are described. There are three anatomical planes generally used in anatomy to describe the human body and structure within the human body: the axial plane 52, the sagittal plane 54 and the coronal plane 56 (see FIG. 3). Additionally, devices and the operation of devices are better understood with respect to the caudad 60 direction and/or the cephalad direction 62. Devices positioned within the body can be positioned dorsally 70 (or posteriorly) such that the placement or operation of the device is toward the back or rear of the body. Alternatively, devices can be positioned ventrally 72 (or anteriorly) such that the placement or operation of the device is toward the front of the body. Various embodiments of the spinal devices and systems of the present invention may be configurable and variable with respect to a single anatomical plane or with respect to two or more anatomical planes. For example, a component may be described as lying within and having adaptability in relation to a single plane. For example, an anchoring device may be positioned in a desired location relative to an axial plane and may be moveable between a number of adaptable positions or within a range of positions. Similarly, the various components can incorporate differing sizes and/or shapes in order to accommodate differing patient sizes and/or anticipated loads.

Turning back to FIG. 2D, a vertebral body 14 is depicted in at least partial cross-section along, for example a sagittal plane 54 and a facet joint 32 is depicted in a coronal plane 56. As will be appreciated, the orientation of a facet joint 32 in any plane of the body changes depending upon the location of a particular joint within the spinal column, this example is provided for illustration purposes only.

The facet joint 32, is formed from a superior articular facet 30 and an inferior articular facet 31. The inferior articular facet 31 has a cephalad facet surface and the superior articular facet 30 has a caudad facet surface. When healthy and normal, each of these surfaces has an articulating cartilage layer positioned adjacent the facet surfaces to improve the movement of the facet joint 32 in operation. In addition to the caudad facet surface and the cephalad facet surface that comprise the opposing joint surfaces, each of the superior articular facet 30 and the inferior articular facet 31 may have additional surfaces on the sides of the facets. A facet capsule 86 is also provided that surrounds the facet joint 32 and to communicate with the various surfaces on the sides of the superior articular facet 30 and the inferior articular facet 31. Where the anatomic or functional manifestations of a disease has resulted in a spinal pathology, facet joint degradation can occur, which includes wear of the articulating surface of the facet joint. Normally, the peripheral, cortical rim of the joint is not affected, or is minimally affected. With hypertrophic facets, the mass of cortical bone and action of the osteophytes can make the facet larger than normal as the facet degenerates. When a facet begins to wear, the biomechanics of the functional spine unit are altered, which can cause further damage to the facet joint as well as pain. Moreover, such alteration of the biomechanics can compromise the integrity of the remainder of the functional spinal unit, and lead to intervertebral disc degradation and damage, further facet joint degradation and damage, spondylolithesis and/or reductions/changes in disc height, as well as the potential occurrence of spinal stenosis (all of which could occur not only in the affected spinal level, but in other spinal levels as well).

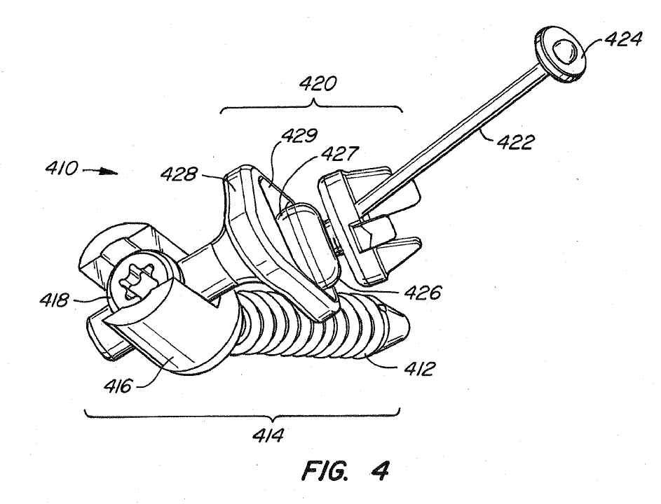

FIG. 4 depicts an embodiment of facet replacement device according to the invention. The device is an artificial facet joint replacement device 410 configured to replace a portion of a natural facet joint. The device 410 comprises fixation elements 412 and 422 that connect the device 410 to the corresponding vertebral structures supporting the caudad and cephalad components of the natural facet joint. In this embodiment, the caudad component of the device 410 incorporates a screw or stem as the fixation element 412 that connects into or near the pedicle of the caudad vertebral body. An adjustable connection 414 connects the base 416 of the fixation element 412 to the artificial caudad joint 428, allowing adjustment and/or rotation of the artificial caudad joint 428 along and/or around one or more axes relative to the fixation element 412. Desirably, the construction is adapted and configured to permit continuous adjustment through relative rotation of the facet joint element and the fixation element around and/or along many different axes through an adjustability range, up to a motion limit provided by a limit stop (if any). In other embodiments, however, the number of axes of rotation may be limited, and the movement may be permitted only in discrete increments. In various embodiments the facet joint element may be moved medially, laterally, superiorly and/or inferiorly with respect to the fixation element. The cephalad fixation element 422 comprises a cable which can be securable through, for example, the lamina or spinous process (FIGS. 2A-D, 22) by use of an anchor 424. Desirably, the use of a flexible cable allows for varying alignment of the cable relative to the artificial cephalad joint 426. For example, the surgeon may wish to create an opening through the lamina and/or spinous process which is optimal for fixation strength of the anchor, but which does not extend along a pre-determined longitudinal axis of the artificial cephalad joint 426. In such a case, the actual orientation of the opening relative to a desired position of the artificial cephalad joint could of infinite variability, which can easily be accommodated by the flexible cable. Desirably, the cable will draw the artificial cephalad joint into intimate contact with the outer surface of the cephalad vertebral body, securing the joint to the vertebral body and rendering the joint capable of immediately bearing load (if desired) as well as facilitating fixation and/or bony ingrowth into the joint. To properly orient the artificial cephalad joint, the surface of the cephalad vertebral body may be pre-shaped such that, when the artificial cephalad joint is in intimate contact with the targeted vertebral surface, it mates with the pre-shaped surface and thus occupies a desired position and/or orientation. Alternatively, a series of differently sized and/or shaped artificial cephalad joints could be provided. A cephalad bearing 426 is connected to the artificial cephalad joint, the bearing 426 having a surface 427 which is adapted to interact with an opposing surface of the artificial caudad joint 428. If desired, a series of cephalad bearings of differing shapes, sizes and/or orientations and/or lengths can be provided to accommodate different objectives, including alteration of bearing height/orientation relative to the fixation element, to accommodate different loading conditions due to other surgical treatments (i.e., artificial disc replace of the same or other spinal level, annular repair, nucleus replacement, dynamic stabilization, interspinous spacer and/or adjacent level fusion devices). The artificial caudad joint is configured to present a surface 429 that receives the surface 427 of the cephalad bearing 426. Thus, for example, forming mating convex/concave surfaces to facilitate movement of each component of the facet joint element 420. The artificial caudad joint is further adapted to engage the base 416 of the fixation element 412 such that the artificial caudad joint can be adjusted during and/or after implantation of the fixation element and then locked in place using a base anchor 418. The outwardly facing surface of the base anchor 418 can be adapted to engage, for example, a driver, such as a flathead screwdriver, a Phillips head screwdriver, or a hexalobe driver. The base anchor 418 is further adapted to secure the artificial caudad joint 428 to the base 416 of the adjustable connector 414. While the fixation element 412 of the polyaxial connector 414 is depicted as incorporating threads to secure it to the vertebral body, it should be understood that a host of other fixation mechanisms, including textured/bony in-growth surfaces, expanding anchors, clamps and/or adhesives can be used.

The relative locations and/or orientations for the fixation elements 412 and 422 of the artificial facet joint 410 are generally mandated by the patient's natural anatomy (such as the locations, orientations and/or conditions of the pedicles, lamina, spinous process and/or vertebral bodies themselves), as well as the objectives of the surgical procedure, and the tissues and structures removed and/or modified during the surgical procedure. Desirably, these fixation elements will be optimally placed for secure fixation. However, optimal placement for secure fixation may not always equate to optimal placement for proper function of the various components of the facet replacement device, and thus there may be a need to adjust and/or alter the position(s) and/or orientation(s) of the artificial cephalad and caudad joints 426 and 428 relative to their respective fixation elements (which desirably can be accommodated by the previously-described adjustability of the caudad and cephalad components). Accordingly, after implantation and adjustment, the artificial caudad joint 428 and the artificial cephalad joint 426 may be in an anatomically correct position within the patient's body (relative to the anatomical structures they augment and/or replace) or in an non-anatomically correct position, depending on the desired clinical outcome and the condition of the spinal anatomy (including, e.g., whether the vertebra have been anatomically altered, either surgically or naturally), as well as any other considerations and requirements of the situation.

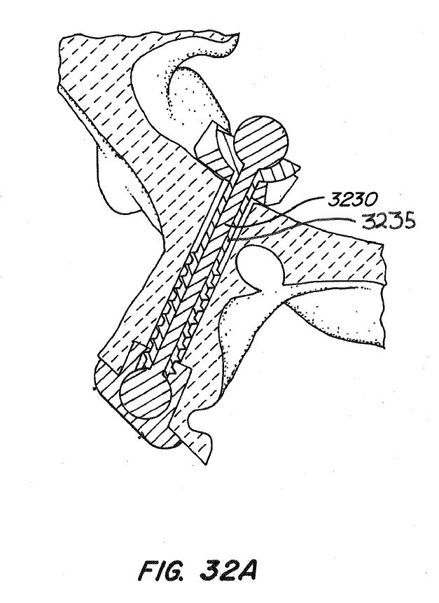

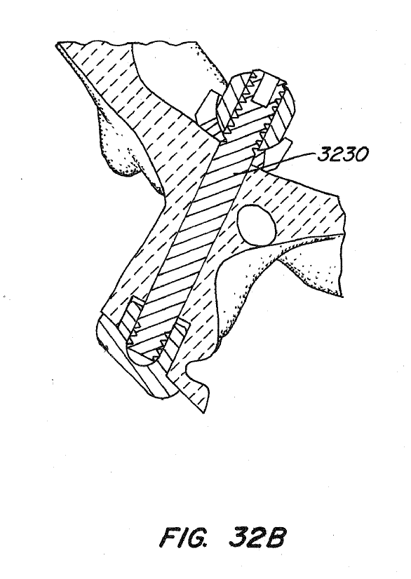

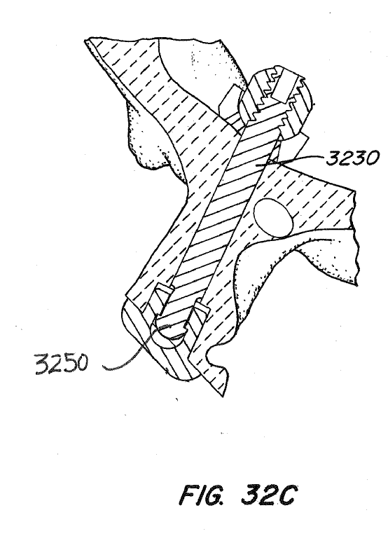

In one alternate embodiment, the fixation element could comprise a cable 3230 and cannulated tube 3235 (see FIG. 32A), with the cannulated tube passing through the targeted bony structure of the lamina and/or spinous process and encircling the cable along at least a portion of its length. The cable 3230 would desirably facilitate flexibility of the implanted artificial facet joint 3100, while the cannulated tube would, among other things, significantly increase the cross-sectional surface area of the cable, thereby reducing the opportunity of the cable to “pull-out” of the bone laterally. A bearing engages one end of the cable and an anchor engages the opposing end. Alternatively, the fixation element could comprise a solid threaded rod 3230 (see FIG. 32B) or a solid rod having a flexible or adjustable engagement member 3250 (see FIG. 32C) allowing for some variation between the orientation of the cephalad bearing and the longitudinal axis of the rod. In these embodiments, the cross-section of the cable/rod is desirably of a sufficient size to prevent the force applied by the cable in the lamina from exceeding the ability of the lamina to resist the force; i.e., a cable of insufficient diameter could present a force great enough to tear through the lamina. Moreover, the cross-section of the cable/rod need not be circular, but may be any shape, including irregular shapes, to desirably present a surface to the surrounding bone that is (1) large enough (and/or flat enough) to resist “pull-out” through the bone (e.g. along the length of the cable), (2) non-circular to resist rotation of the cable/rod, and/or (3) of increased surface area to facilitate bony in-growth into the cable/rod. The anchors can further be adapted to provide internal threads such that the anchors can be secured to the cable by screwing the anchor onto a threaded end of the cable, or can be adapted to provide an aperture that enables the anchor to be snap fit onto the end of the cable. Other configurations will be apparent to those skilled in the art.

The device 410 can be attached to a cut or resected portion of the vertebra. The cephalad portion of the device 410 is secured to the vertebral via an extension cable 422 that passes through the lamina (see, 20 of FIG. 2A) while the caudad portion of the device is secured to the vertebra by, for example, a pedicle anchor. As will be appreciated by those skilled in the art, the bearing surfaces 427, 429 may be flat, spherical or a range of concavities/convexities and could be used in varying combinations. The bone-interfacing surface of the device can be configured to consist of any of a variety of surfaces that prevent relative motion and/or promote bone in-growth. Additionally, a pocket, or recess, can be provided to deliver substances to stimulate local bone growth (for example, BMPs, bone graft, bone graft substitutes, etc.) as well as to serve as a relative motion limiter once bone growth into the recess or pocket has occurred. Moreover, the cable 422 enables the device to achieve optimal angular and depth flexion. Further choosing the length of the cable 422 can further optimize the performance of the device 410 after implantation because the variable length of the cable can account for a wider variety of spinal anatomy. Further, the cable 422 can be tensioned as desired by the surgeon further adapting the device to a particular physiological condition to be corrected.

A portion of the vertebra may be surgically removed or altered, for example, to permit access to and removal of the superior facet of the caudad vertebra. A pedicle anchor 412 is then deployed and the caudad bearing is affixed to the anchor. Adjustability of the implanted device 410 may be achieved through the use of varying sized components as well as altering configuration and fit (e.g., with the use of polyaxial anchors, tapers, interlocking splines, etc.). Implantation of the device can also be accomplished using minimally invasive surgical techniques. Repair, replacement and/or augmentation can also be performed using limited-open, modified-open, and/or fully open surgical procedures. For example, where facet joint replacement is necessary, but removal of soft and/or hard tissue in and/or adjacent the spinal canal is not warranted or desired (such as where spinal stenosis and nerve impingement is not a significant concern), the repair and/or replacement of one or more facet joints can be accomplished in a least-invasive fashion using one or more cannulae to implant the device and associated hardware. Alternatively, where the removal of the facet joint 32 and/or lamina 20 is necessitated (shown in FIG. 2), such a procedure can be accomplished through a combination of open, semi-open, and/or minimally invasive procedures to minimize damage and/or disruption to surrounding soft-tissue structures, In such a procedure, one or more of the facet joint capsules can be exposed through an open incision or semi-open procedure such as through an expanding cannula (to allow easy resection and removal of the facet joint, allow easy introduction of larger components of the facet joint, and/or surrounding anatomical structures), and the cephalad component of the facet replacement can be delivered through the lamina through a cannula or other minimally-invasive delivery method.

Another advantage of the embodiment is that the device is positioned within the lamina with only limited portions of the implant extending outwards from the vertebral body. This arrangement presents a low-profile to the surrounding soft tissue structure, resulting in less interaction between the device and the surrounding soft tissues, as well as less displacement of natural tissues due to the presence of the implant. Moreover, anchoring the cephalad portion of the device within the lamina and/or spinous process reduces and/or eliminates the opportunity for unwanted contact between the dura and the implanted device. Desirably, once the facet replacement components are implanted, the device will induce a healing response in the patient's body, causing formation of a capsule or pseudo-capsule of soft tissue (including scar tissue) around the articulating elements of the facet replacement prosthesis.

Further, based on the position of the caudad bearing surface, a translaminar aperture or hole can be placed through a percutaneous cephalad approach targeting the bearing surface. Through a caudad approach (which may be similarly minimally-invasive, limited open, open or through an expanding cannula), the cephalad bearing portion of the device is placed in a desired position/orientation and the tension cable 422 is drawn in a retrograde fashion through the translaminar opening formed in the lamina or spinous process. The cable is then tightened and secured to the superior aspect of the lamina 20. Once the cable 422 is secured and the superior aspect of the lamina, the cephalad portion of the device is secured against the cut surface of the inferior facet. Using such a minimally invasive surgical approach, the posterior structures, except for the facet and facet capsules, can be left undisturbed.

As will be appreciated upon reviewing the entire specification, the device 410 will also work in conjunction with a total disc replacement. Use with a total disc replacement provides a solution for the total disc replacement contraindication of facet degeneration. Implantation of a total disc replacement device prior to implanting the facet restoration device 410 opens the disc space, aiding in any decompression of the joint that may be necessary. The device may be used unilaterally or bilaterally, depending on the nature of and stage of disease, and can be used at multiple levels of the spine. Similarly, removal of some or all of the facet structures (and lamina, etc.) of the targeted vertebral level may permit the passage of one or more components of the artificial disc replacement (or nucleus replacement, or annular repair material, and their respective tools) through the removed facet tissues via a lateral, posterior-lateral and/or posterior approach. The functions of the removed tissues can then be replaced by implanting the facet replacement prosthesis as described herein.

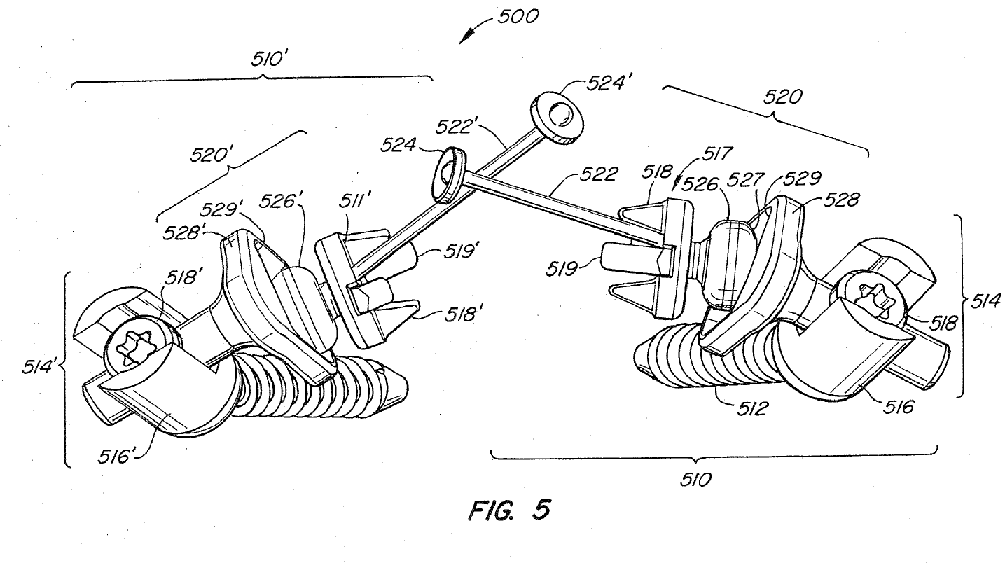

FIG. 5 illustrates a bilateral facet replacement system 500 similar to the facet replacement device of FIG. 4 illustrated as a bilateral solution (e.g., treating a facet joint on either side of the midline of the spine). The device is an artificial facet joint replacement device 510, 510′ configured to replace a portion of a natural facet joint and a fixation element 512, 512′ that enables the device 510, 510′ to connect to a facet joint via, for example, an adjustable connection 514, 514′. The connection 514, 514′ permits artificial caudad joints 528, 528′ 520, 520′ and caudad bases 516, 516′ to be adjusted with respect to the fixation elements 512, 512′ about more than one axis. As discussed above, the construction can be adapted and configured to permit continuous adjustment through relative rotation of the facet joint element and the fixation element around many different axes through an adjustability range, up to a motion limit provided by a limit stop. In other embodiments, however, the number of axes of rotation may be limited, and the movement may be permitted only in discrete increments. In various embodiments the facet joint element may be moved medially, laterally, superiorly and/or inferiorly with respect to the fixation element. The facet joint element 520, 520′ is further comprised of a flexible or inflexible rod or cable 522, 522′, which is securable through, for example, the lamina and/or spinous process by use of an anchor 524, 524′. The cable 522, 522′ is adapted to engage an artificial cephalad joint 526, 526′ having a surface 527, 527′ adapted to mate with an opposing surface of an artificial caudad joint 528, 528′. The artificial caudad joint is configured to present a surface 529, 529′ that received the artificial cephalad joint. Thus, for example, forming mating convex/concave surfaces to facilitate movement of each of the cephalad and caudad components 526, 528 of the facet joint element 520, 520′. The artificial caudad joint is further adapted to engage the caudad base 516, 516′ of the fixation element 512, 512′ such that the artificial caudad joint can adjusted during implantation and then locked in place using a base anchor.

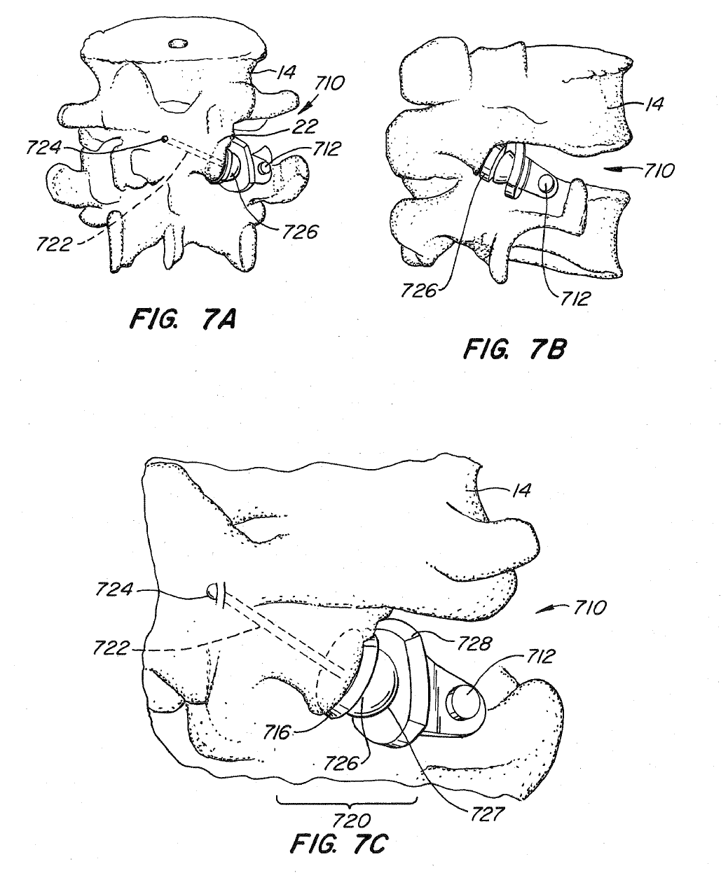

The relative positions of facet joint 510, 510′ and fixation element 512, 512′ may be set prior to implant, after implant, OT both before and after implant. After implant and adjustment, the artificial caudad joint 528, 528′ and the artificial cephalad joint 526, 526′ may be in an anatomically correct position within the patient's body or in an non-anatomically correct position, depending on the desired clinical outcome and/or the condition of the spinal anatomy (e.g., whether the vertebra have been anatomically altered, either surgically or naturally), as well as any other considerations and requirements of the situation. In this embodiment, the cephalad component of the device is secured to the lamina (shown in FIGS. 2A-D, 20) of the cephalad vertebral body (e.g., 14 of FIG. 2B) using flexible cables or rods 522, 522′. The cables 522, 522′ can be configured to pass through some or all of the laminar surface, as will be better appreciated with respect to, for example, FIG. 7. The inner surface 511, 511′ of the cephalad facet joint component 526, 526′ can be further adapted and configured to incorporate a textured, biologic or bony in-growth surface which promotes and/or allows biological in-growth, thereby augmenting attachment of the component of the surface to the vertebral body. Augmentation suitable for bony in-growth can be provided using, for example, resorbable bone cement, which increases the strength of the surface. The device can also be used in combination with bone filler or allograft material. Suitable bone filler material includes, the use of bone material derived from demineralized allogenic or xenogenic bone and can contain substances for example, bone morphogenic protein, which induce bone regeneration at a defect site. See, U.S. Pat. No. 5,405,390 to O'Leary et al. for Osteogenic Composition and Implant Containing Same; U.S. Pat. No. 5,314,476 to Prewett et al. for Demineralized Bone Particles and Flowable Osteogenic Composition Containing Same; U.S. Pat. No. 5,284,655 to Bogdansky et al. for Swollen Demineralized Bone Particles, Flowable Osteogenic Composition Containing Same and Use of the Compositions in the Repair of Osseous Defects; U.S. Pat. No. 5,510,396 to Prewett et al. for Process for Producing Flowable Osteogenic Composition Containing Demineralized Bone Particles; U.S. Pat. No. 4,394,370 to Jeffries for Bone Graft Material for Osseous Defects and Method of Making Same; and U.S. Pat. No. 4,472,840 to Jeffries for Method of Inducing Osseous Formation by Implanting Bone Graft Material, which disclose compositions containing demineralized bone powder. See also U.S. Pat. No. 6,340,477 to Anderson for Bone Matrix Composition and Methods for Making and Using Same, which discloses a bone matrix composition.

One or more prongs or projections 518, 519 are positioned on the inner surface of the base 517 of the cephalad facet joint component to prevent rotation and/or secure the component to the lamina. For example, the projections 518 are positioned such that they penetrate the laminar surface while the flattened projections 519 are positioned to desirably lay adjacent or against the outer surface of the lamina. As will be appreciated by those skilled in the art, the laminar surface can be prepared prior to the implanting the device, e.g., through resection of the articulating facet surface and/or the laminar surface, to provide a surface which, when abutting against the cephalad component will properly orient and position the cephalad component relative to an adjoining caudad bearing component. The cephalad facet joint component 526 has a bearing surface 527 that engages a bearing surface 529 of a caudad facet joint component 528.

The components can be secured into and through the pedicles of the vertebral body. A multi-axial or poly-axial anchor can be used to permit in situ adjustment of the caudad bearing surface. Alternatively, an adjustable component can be used that permits adjustment. As will be appreciated by those skilled in the art, other anchors can be employed without departing from the scope of the invention.

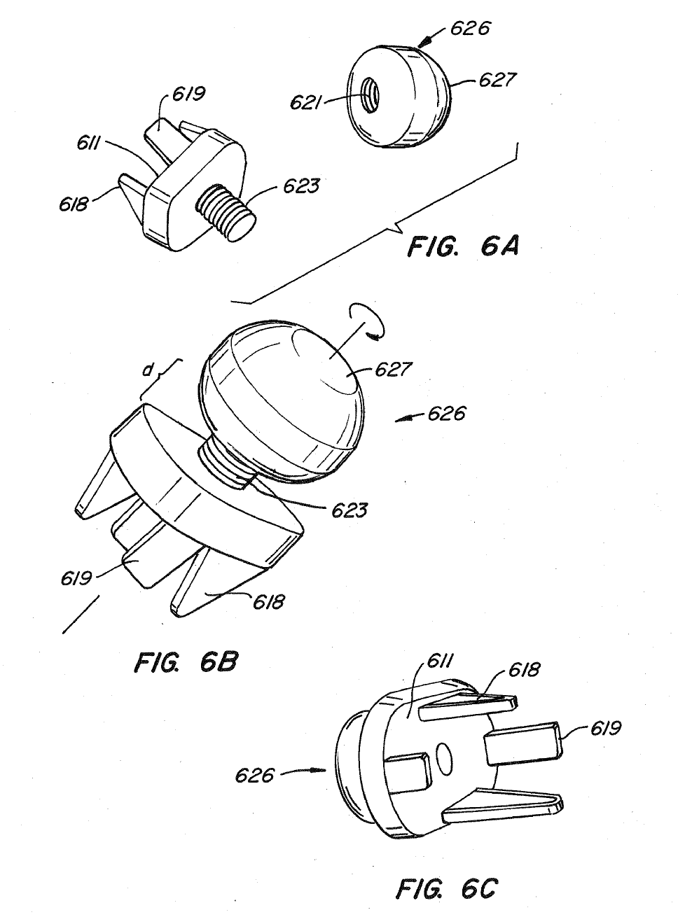

FIG. 6A illustrates two components of the cephalad facet joint portion of the facet replacement system shown in FIGS. 4-5. One or more prongs or projections 618, 619 are positioned on the inner surface 611 of the cephalad facet joint component to prevent rotation and/or secure the component to the lamina of a vertebral body. The artificial cephalad joint component 626 has a bearing surface 627 that is adapted to enable the surface to engage a mating caudad joint surface. The artificial cephalad joint component 626 is configured to be removable from a base element by use of an aperture 621 on the joint component and threads 623 on the base component. The distance d between the upper surface of the joint component and the surface of the base component can be adjusted by controlling the rotating the joint element about the threads 623 on the base component. Thus, depending upon how far down onto the neck of the threaded base member the joint component is turned will effect the overall height of the device. Thus adaptability enables the device to be adjusted in situ to provide a better anatomical fit within a particular patient. FIGS. 6B-C illustrate the two components illustrated in FIG. 6A in combination from different perspectives. As an alternative, a series of artificial cephalad joint components 626 of differing shapes, sizes and/or orientations and/or lengths can be provided to accommodate different objectives, including alteration of bearing height/orientation relative to the fixation element, to accommodate different loading conditions due to other surgical treatments (i.e., artificial disc replacement of the same or other spinal level, annular repair, nucleus replacement, dynamic stabilization, interspinous spacer and/or adjacent level fusion and/or facet replacement devices). Moreover, to accommodate differing designs (i.e., constrained discs versus unconstrained discs) and/or arrangement/positioning of artificial disc replacement devices used on the same or different spinal levels, the artificial cephalad joint components 626 (and their respective artificial caudad joint components) could be of differing shapes, sizes, orientations and/or lengths to accommodate the different loading profiles induced or desired by the artificial disc replacement devices.

FIGS. 7A-C depict an alternative embodiment of a facet replacement device, similar to those depicted in FIGS. 4-6, implanted within a vertebral body 14. As shown in FIG. 7, during implantation, the cephalad facet surface can be prepared and then the proximal end of the flexible cable or rod 722 is secured to the vertebral body. As can be seen from FIG. 7, the device 710 as implanted is configured to replace a portion of a natural facet joint. The fixation element enables the device 710 to connect to a facet joint. The facet joint element 720 is further comprised of a flexible rod or cable 722, which is securable through, for example, the lamina and/or spinous process 22 by use of an anchor 724. The cable 722 is adapted to engage an artificial cephalad joint 726 having a surface 727 adapted to mate with an opposing surface of an artificial caudad joint 728. During a surgical implantation, the cephalad facet surface of the vertebral body can be prepared prior to implantation of the device. Thereafter, the proximal end of the flexible cable or rod is inserted into and through the lamina/spinous process in a desired direction and orientation with a first end of the cable 722 attached to the artificial cephalad joint component 726 and the second end adapted to engage an anchor 724 or cable lock. The anchor, or other securing device (not shown), can be attached (e.g., threaded) onto the end of the cable 722 and abutted against the lamina 20 of the vertebral body 14. A tensioning tool and/or crimper, or similar device, can be used to tension the cable, thereby drawing the projections of the caudad base 716 toward the laminar surface. As will be appreciated by those skilled in the art, as the base is drawn toward the laminar surface, the projections (shown in FIGS. 4-6) may be drawn into and/or against the lamina, depending upon the actual configuration of the projections. Thereafter, once a desired tension has been reached, the device can then be secured by locking the cable with the anchoring device 724. Excess cable can be resected, if desired. As described above with respect to FIG. 6, the cephalad component (as well as the respective caudad component) can be configured to provide an adjustable and/or removable bearing surface. Thus, the bearing surface location can be tailored to achieve the performance needs for the facet replacement device for a particular patient.

The caudad component 728 can also be secured into and through the pedicles of the caudad vertebral body. As illustrated in this embodiment, the caudad component is secured to the vertebral body by use of a fixation element 712.

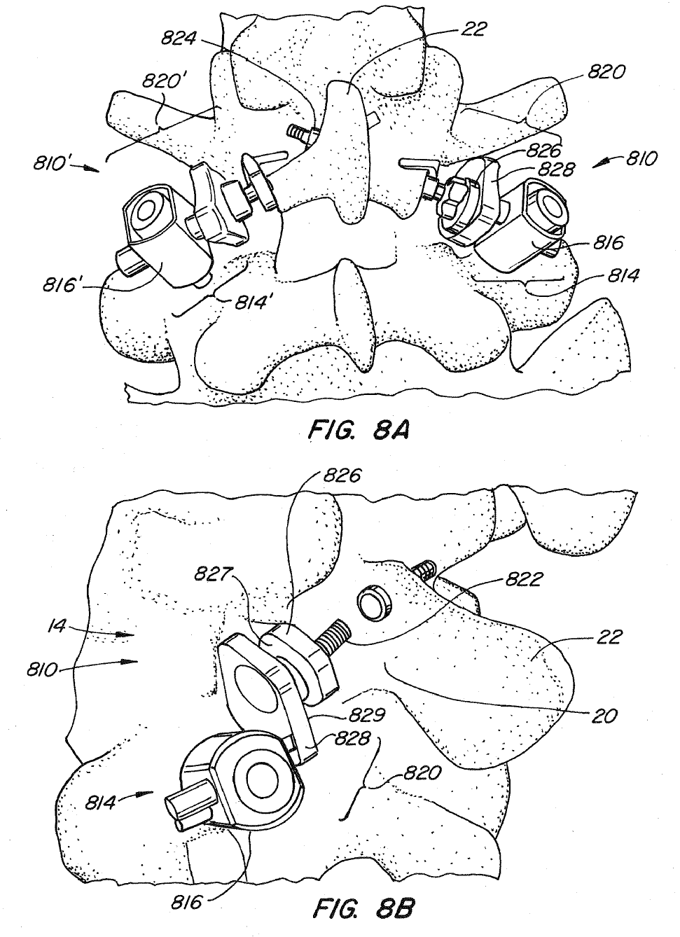

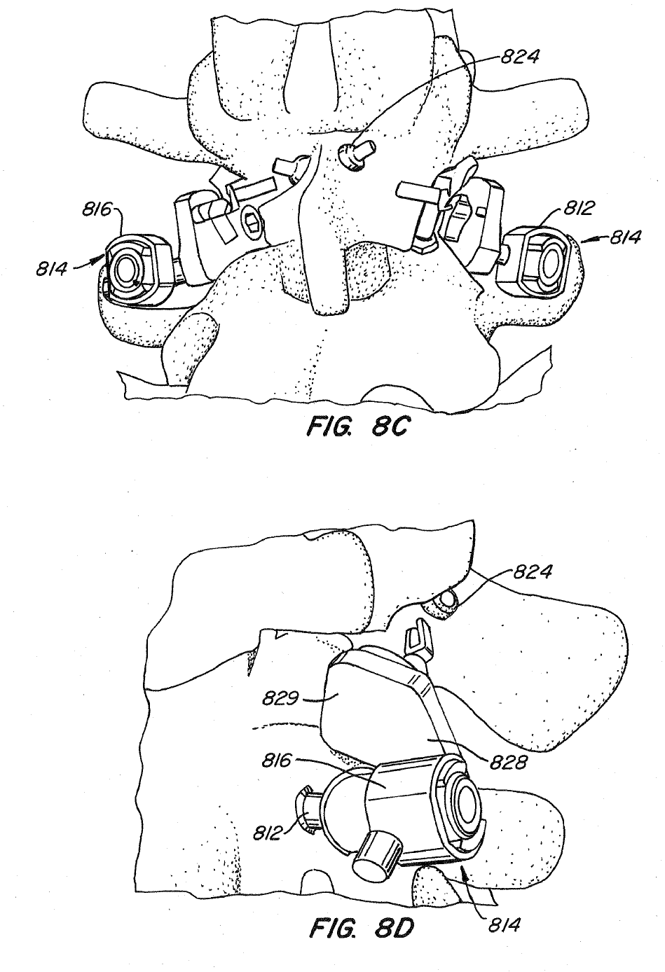

FIGS. 8A-D illustrate an implanted facet replacement device according to another embodiment of the invention. The device 810 as implanted is configured to replace a portion of a natural facet joint. The fixation element enables the device 810 to connect to a facet joint via, for example, a polyaxial connection 814. The connection 814 permits the artificial caudad joint 828 and caudad base 816 to be adjusted with respect to the fixation element 812 around more than one axis within the patient. As can be appreciated by reviewing FIG. 8, the facet joint element may be moved medially, laterally, superiorly and/or inferiorly with respect to the fixation element attached to the vertebral body. The facet joint element 820 is further comprised of an anchoring stem 822, which is securable through, for example, the lamina and/or spinous process 22 by use of a lock 824. The anchoring stem 822 is adapted to engage an artificial cephalad joint 826 having a surface 827 adapted to mate with an opposing surface 829 of an artificial caudad joint 828. During a surgical implantation, the cephalad facet surface of the vertebral body can be prepared prior to implantation of the device. Thereafter, the proximal end of the flexible cable or rod is inserted into and through the lamina/spinous process in a desired direction and orientation with a first end of the anchoring stem 822 attached to the artificial cephalad joint component 826 and the second end adapted to engage an anchor 824 or cable lock. The anchor, or other securing device, can be attached (e.g., threaded) onto the end of the anchoring device 822 and abutted against the lamina 20 of the vertebral body 14. A tensioning tool and/or crimper, or similar device, can be used to provide sufficient torque to lock the anchor 824 onto the anchoring stem 822, thereby drawing the projections of the cephalad base 817 toward the laminar surface. As discussed above, as the base is drawn toward the laminar surface, the projections may be drawn into and/or against the lamina, depending upon the actual configuration of the projections. Thereafter, once a desired tension has been reached, the device can then be secured by locking the anchoring stem 822 with the anchoring device 824. As described above with respect to FIG. 6, the cephalad component (as well as the respective caudad bearing surface) can be configured to provide an adjustable and/or removable bearing surface. Thus, the bearing surface(s) can be tailored to achieve the performance needs for the facet replacement device for a particular patient. As further described above, the caudad component in this embodiment is configured to include a multi-axial or poly-axial component that is adjustable to permit in situ adjustment of the caudad bearing surface.

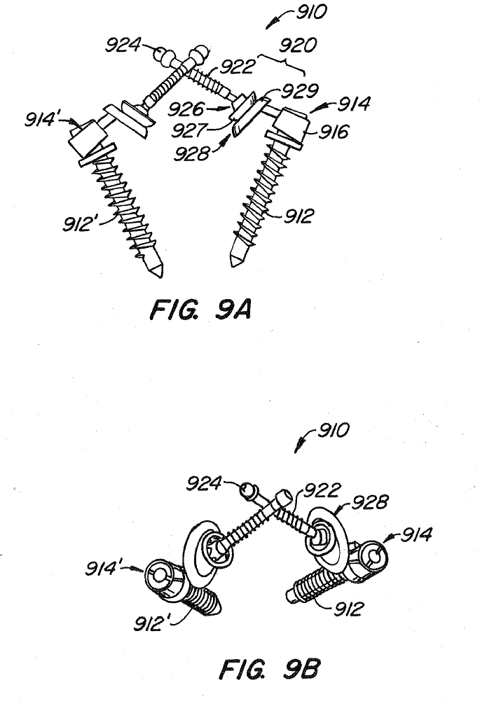

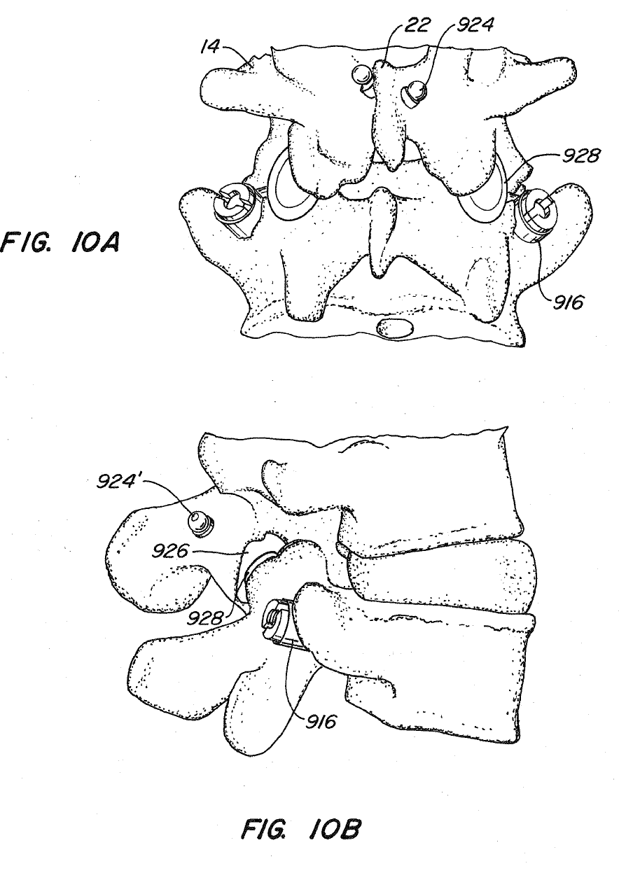

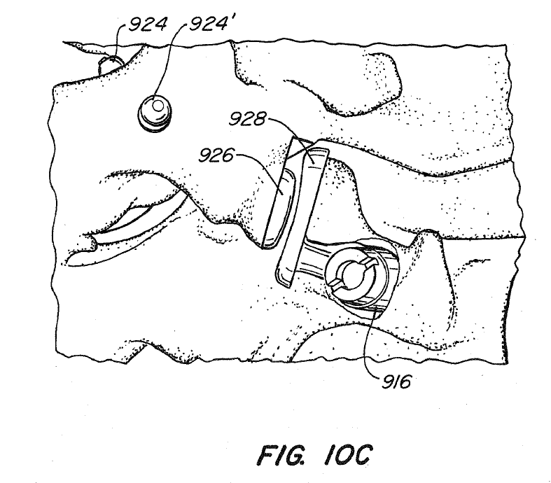

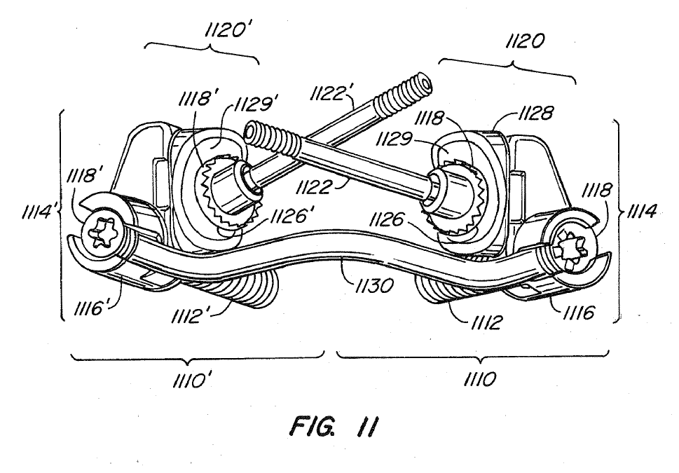

FIGS. 9A-B illustrate a facet replacement device according to another embodiment of the invention from a side view and a top view. The device 910 can be implanted to replace a portion of a natural facet joint. The fixation element 912 enables the device 910 to connect to a facet joint via, for example, an adjustable (e.g., polyaxial) connection 914. The connection 914 permits artificial caudad joint 928 and caudad base 916 to be rotated with respect to the fixation element 912 around more than one axis within the patient, if desired. As can be appreciated by reviewing FIG. 9, the facet joint element may be moved medially, laterally, superiorly and/or inferiorly with respect to the fixation element attached to the vertebral body. The facet joint element 920 is further comprised of a cephalad threaded anchoring stem 922, which is securable through, for example, the lamina and/or spinous process 22 and secured at a proximal end by a lock 924. The anchoring stem 922 is adapted to engage an artificial cephalad joint 926 having a surface 927 adapted to mate with an opposing surface 929 of an artificial caudad joint 928. The caudad joint 928 is configured to engage the threaded anchoring stem. Thus, when the anchoring stem is secured to the vertebra, the position of the caudad joint surface 929 can be adjusted to optimize the position of the caudad joint relative to the cephalad joint.

As with previous embodiments, during a surgical implantation, the cephalad facet surface of the vertebral body can be prepared prior to implantation of the device. Thereafter, the proximal end of the cable or rod (which may be flexible or non-flexible) is inserted into and through the lamina and/or spinous process in a desired direction and orientation with a first end of the anchoring stem 922 attached to the artificial cephalad joint component 926 and the second end adapted to engage an anchor 924 or cable lock. The anchor, or other securing device, can be attached (e.g., threaded) onto the end of the anchoring device 922 and abutted against the bony surface or lamina 20 of the vertebral body 14. A tensioning tool and/or crimper, or similar device, can be used to provide sufficient torque to lock the anchor 924 onto the anchoring stem 922 (if desired), thereby drawing the projections of the base 916 toward the laminar surface. As discussed above, as the base is drawn toward the laminar surface, the projections may be drawn into and/or against the lamina, depending upon the actual configuration of the projections. Thereafter, once a desired tension has been reached, the device can then be secured by locking the anchoring stem 922 with the anchoring device 924. Desirably, this arrangement will allow much of the loading experienced by the artificial cephalad joint component 926 to be compressive in nature, thereby allowing transfer of a significant amount of these forces directly into the laminar surface, rather than to the cable or rod 922. As described above with respect to FIG. 6, the cephalad component can be configured to provide an adjustable and/or removable bearing surface. Thus, the bearing surface location can be tailored to achieve the performance needs for the facet replacement device for a particular patient. As further described above, the caudad component in this embodiment is configured to include an adjustable, multi-axial or poly-axial component that is adjustable to permit in situ adjustment of the caudad bearing surface.

FIGS. 10A-C illustrate the facet replacement device of FIGS. 9A-B implanted from a posterior and lateral perspective. In this embodiment, the caudad attachment is notched on the facet surface. The notching enables the device to engage a prepared or resected surface when implanted.

Turning now to, FIG. 11 a bilateral facet replacement system with a caudad cross-bar is illustrated. The device includes a pair of artificial facet joint replacement devices 1110, 1110′ configured to replace a portion of a natural facet joint on either side of a vertebral body, which includes fixation elements 1112, 1112 that enable the devices 1110, 1110′ to connect to a caudad vertebral body via, for example, a polyaxial connector 1114, 1114′. A cross-bar 1130 is provided connecting each of the caudad components of the artificial facet joint replacement devices 1110, 1110′ at their respective adjustable connectors 1114, 1114′. The cross-bar is formed of a bar having a plurality of curves enabling it to avoid disrupting portions of the spinal anatomy when implanted. Implantation of the cross-bar can be accomplished in a minimally-disruptive fashion by making a small vertical incision in the interspinous ligaments on the targeted spinal motion segment, and then threading the cross-bar through the opening created therein. Desirably, the cross-bar will prevent rotation of the caudad components and permits load sharing between their respective anchors.

As discussed above, the construction can be adapted and configured to permit continuous adjustment through relative rotation of the facet joint element and the fixation element around many different axes through an adjustability range. In other embodiments, however, the number of axes of rotation may be limited, and the movement may be permitted only in discrete increments. In various embodiments the facet joint element may be moved medially, laterally, superiorly and/or inferiorly with respect to the fixation element. The facet joint elements 1120, 1120′ are further comprised of an anchoring stem 1122, 1122′, which is securable through, for example, the lamina and/or spinous process. The anchoring stems 1122, 1122′ are adapted to engage an artificial cephalad joint 1126, 1126′ having a surface adapted to mate with an opposing surface of the artificial caudad joint 1128, 1128′. The artificial caudad joint is configured to present a surface that received the artificial cephalad joint. Thus, for example, forming mating convex/concave bearing surfaces enables the movement of each of the respective cephalad and caudad components 1126, 1128 of the facet joint element 1120 to occur more smoothly. The artificial caudad joint is further adapted to engage the base 1116, 1116′ of the fixation element 1112, 112′ such that the artificial caudad joint can adjusted during implantation and then locked in place using a base anchor.

The relative positions of each of the facet joints 1110, 1110′ and fixation elements 1112, 1112′ may be set prior to implant, after implant, or both before and after implant. After implant and adjustment, the artificial caudad joint 1128, 1128′ and the artificial cephalad joint 1126, 1126′ may be in an anatomically correct position within the patient's body or in an non-anatomically correct position, depending on the desired clinical outcome and/or the condition of the spinal anatomy (e.g., whether the vertebra have been anatomically altered, either surgically or naturally), as well as any other considerations and requirements of the situation. In this embodiment, the cephalad component of the device is secured to the lamina (shown in FIGS. 2A-D, 20) of the cephalad vertebral body (e.g., 14 of FIG. 2B) using an anchoring stem 1122, 1122′. The anchoring stem can be provided with threads 1109, 1109′ that facilitate engaging the bone or adapted to engage an anchor at its end. As will be appreciated by those skilled in the art, the anchoring stem used in this embodiment can be a solid rod, or can be a cable, such as that depicted in FIG. 5, which is configured to pass through some or all of the laminar surface. The cross-bar 1130 is adapted to traverse the midline of the spine when the device is implanted and to engage the base 1116, 1116′ of the connector 1114, 1114′.

The facet replacement components can be further adapted to incorporate artificial ligaments between the articulating arms and/or the treated vertebral bodies. Alternatively, the devices could incorporate a flexible capsule around some or all of the facet/articulating joint surfaces. As will be appreciated by those skilled in the art will appreciate, multiple attachment points can be included, e.g., with the use of apertures, holes, hooks, etc. For attaching existing ligaments, tendons and/or other soft or hard tissues at the conclusions of the surgical procedure to promote healing and further stabilization of the affected level. Moreover, the natural healing response of the body may create a pseudo-capsule of soft and/or scar tissue abound the cephalad and caudad articulating surfaces of the facet replacement device, which may in some manner serve to duplicate some of the functions of the natural facet capsule.

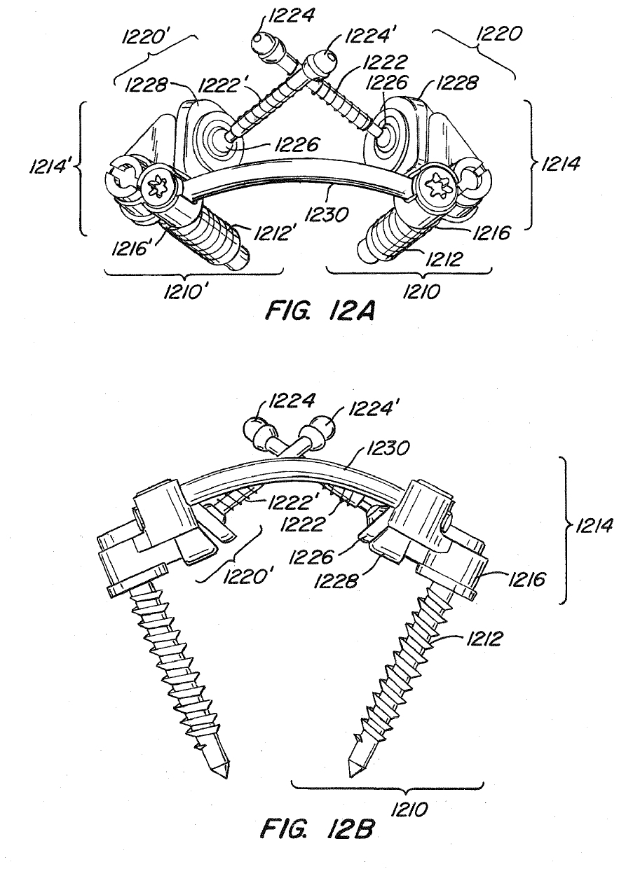

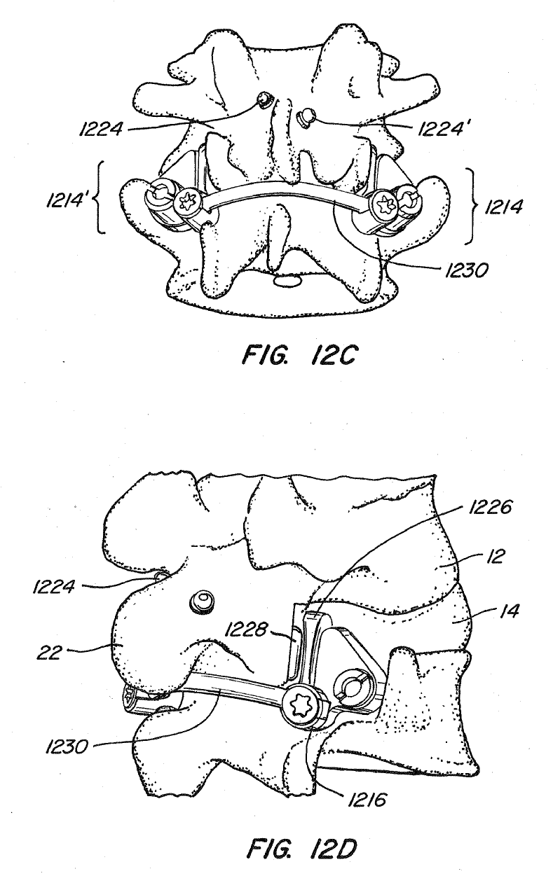

FIGS. 12A-B, a bilateral facet replacement system with a cross-bar is depicted. Similar to the embodiment shown in FIG. 11, the device includes a pair of artificial facet joint replacement devices 1210, 1210′ configured to replace a portion of a natural facet joint on either side of a vertebral body, including a fixation element 1212, 1212 that enables the devices 1210, 1210′ to connect to a caudad vertebral body via, for example, an adjustable connector 1214, 1214′. A cross-bar 1230 is provided connecting each of the artificial facet joint replacement devices 1210, 1210′ at their respective adjustable connectors 1214, 1214′. The cross-bar can be formed of a curved bar that is adapted to follow the exterior curve of the vertebral body. More than one adjustable connector can be provided on each side of the device to enhance the ability of the device to accommodate a wide variety of anatomical differences upon implantation.

Similar to other embodiments, the connector 1214, 1214′ permits the artificial caudad joint 1228, 1228′ and caudad base 1216, 1216′ to be moved and/or rotated with respect to the fixation element 1212, 1212′. The facet joint elements 1220, 1220′ further incorporate anchoring stems 1222, 1222′, which are securable through, for example, the lamina and/or spinous process. The anchoring stems 1222, 1222′ are adapted to engage a artificial cephalad joint 1226, 1226′ having a surface 1227, 1227′ adapted to mate with an opposing surface of the artificial caudad joint 1228, 1228′. The artificial caudad joint is configured to present a surface 1229, 1229 that received the artificial cephalad joint.

As with other embodiments, the relative positions of each of the facet joints 1210, 1210′ and fixation elements 1212, 1212′ may be set prior to implant, after implant, or both before and after implant. After implant and adjustment, the artificial caudad joint 1228, 1228′ and the artificial cephalad joint 1226, 1226′ may be in an anatomically correct position within the patient's body or in an non-anatomically correct position, depending on the desired clinical outcome and/or the condition of the spinal anatomy, as well as any other considerations and requirements of the situation. FIGS. 12C-D illustrates the facet replacement system implanted from the posterior and lateral perspectives.

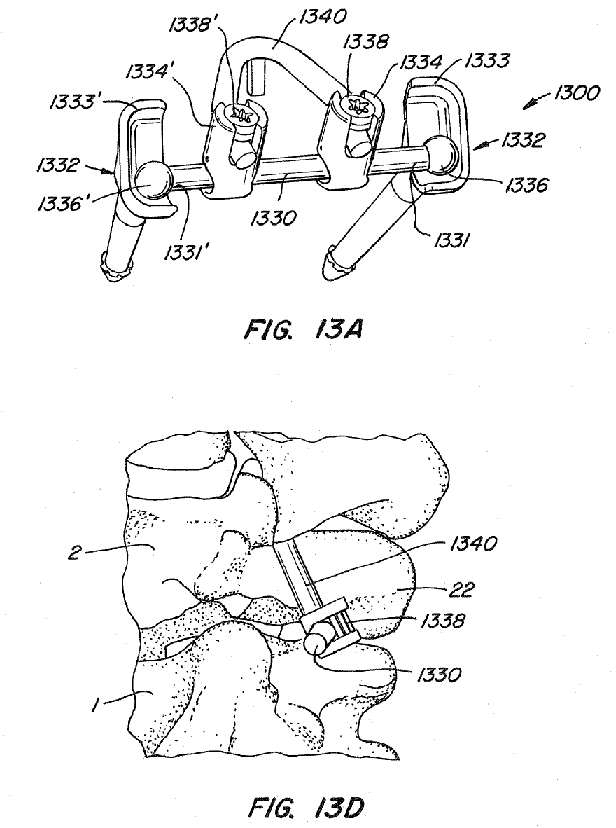

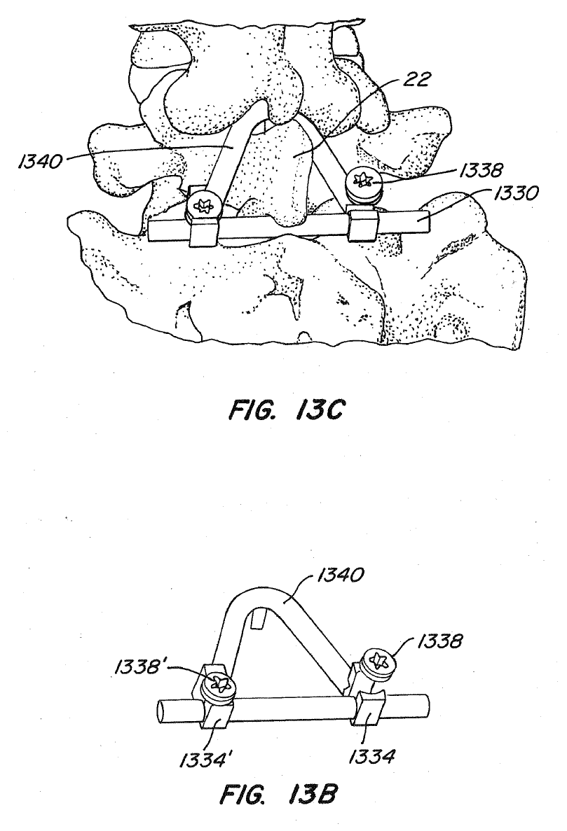

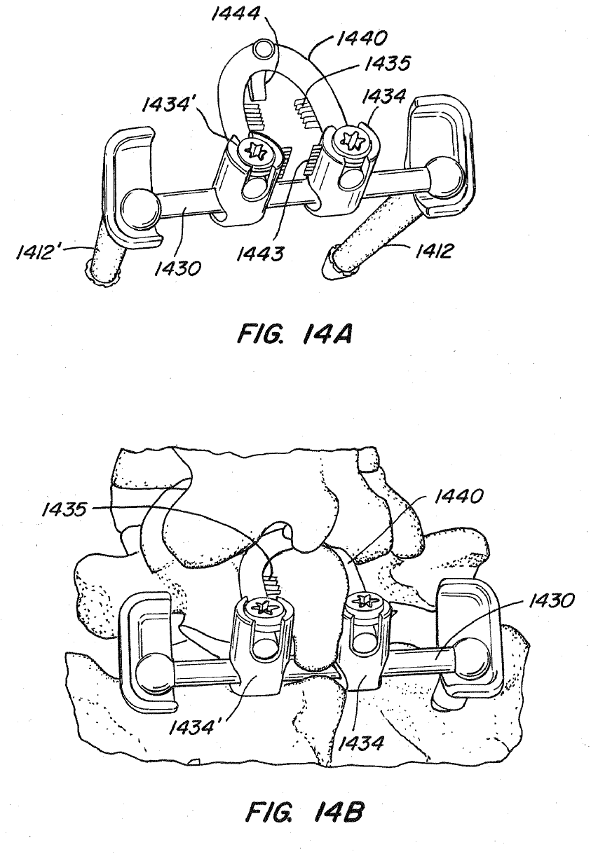

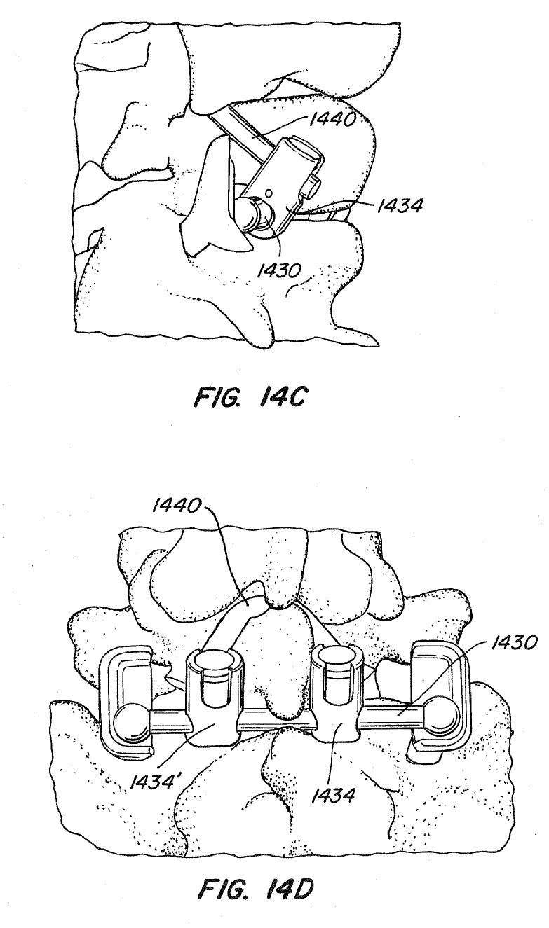

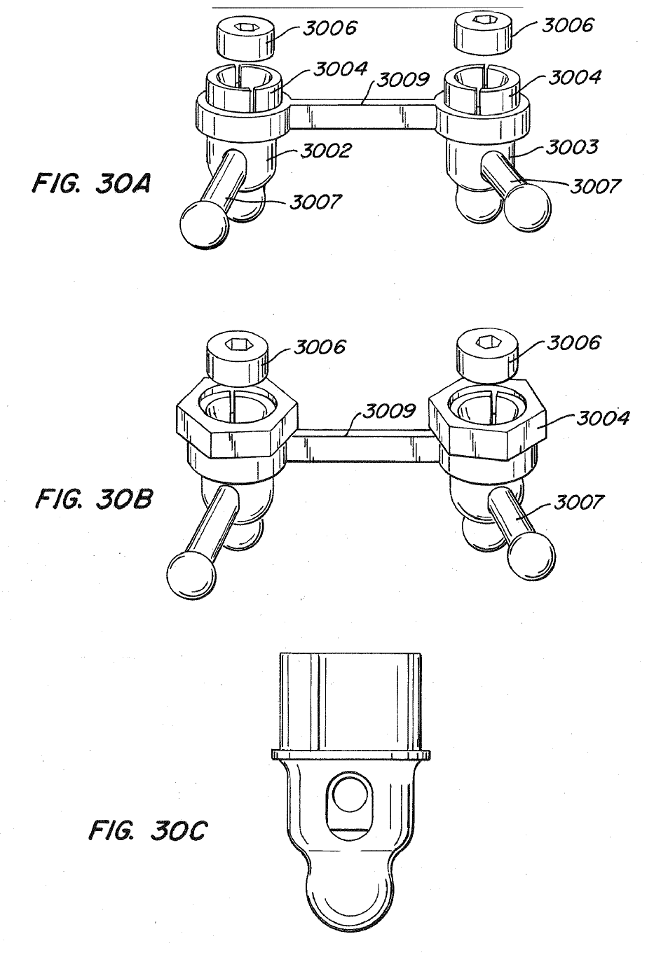

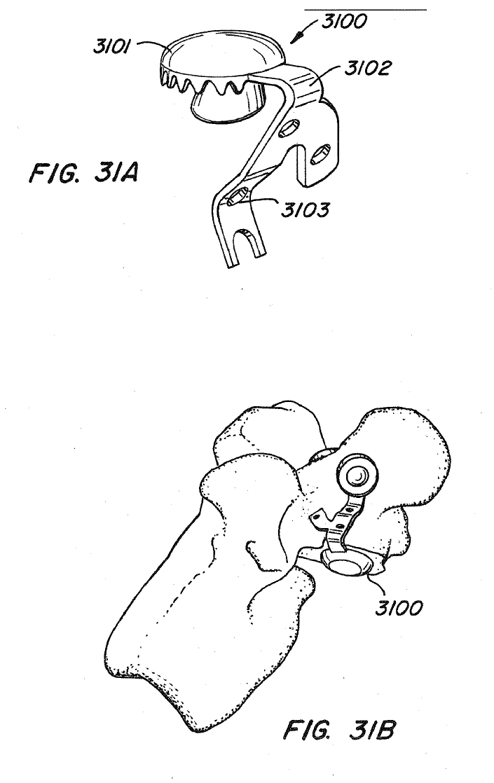

FIG. 13A illustrates another embodiment of a facet replacement system having caudad cups, a cross-bar and a laminar clamp/connection device. The spinal arthroplasty device 1300 includes a crossbar 1330, and a pair of caudad arms 1332, 1332′ having caudad cups 1333, 1333′. In this exemplary embodiment the facets of the spine (see FIGS. 2, 30) are replaced by the cooperative operation of the crossbar 1330, and the adaptable crossbar mounts 1334, 1334′ that engages a laminar clamp 1340 to the crossbar 1330. The crossbar interacts with the caudad arms 1332, 1332′ which form cups 1333, 1333′ to receive the crossbar 1330. The components of the arthroplasty device 1300 are designed to provide appropriate configurability and adaptability for the given disease state, patient specific anatomy and spinal level where the implant occurs.

The crossbar 1330 has a first end 1331 and a second end 1331′. In the illustrated embodiment the crossbar 1330 is a three piece bar where the ends 1331, 1331′ form a threaded male portion. Attached to each crossbar end 1331, 1331′ is an internally threaded ball 1336, 1336′ sized to receive the threads of the cross bar 1330. The threaded ends allow for the width of the crossbar to be adjusted to mate with the width between caudad anchors 1332, 1332′. Additional alternative embodiments of the crossbar 1330 could include a series of solid crossbars of varying widths and/or thicknesses, or an adjustable crossbar having some form of locking or biasing mechanism (such as a spring-loaded tensioner or detent mechanism, etc.).

The crossbar mounts 1334, 1334′ are a connection structure to couple the laminar clamp 1340 to the crossbar 1330. The laminar clamp has ends that extend through a channel in the crossbar mounts 1334, 1334′. In the illustrated embodiment, the crossbar mount 1334, 1334′ includes a laminar anchor engaging portion, a crossbar engaging portion and a fixation element 1338, 1338′. The fixation element 1338, 1338′ anchors the laminar anchor ends with the channel of the crossbar mounts 1334, 1334′. Fixation element can be a screw, stem, cork-screw, wire, staple, adhesive, bone, and other material suitably adapted for the design. As will be described in greater detail below, embodiments of the crossbar mount 1330 provides adaptability.

In the embodiment shown in FIG. 13B, the laminar clamp 1340 connects directly to the cross-bar mounts 1334, 1334′, as opposed to fitting within a channel, and the fixation element 1338, 1338′ anchors the crossbar

FIGS. 13C-D illustrate the clamp portion of the system implanted within a spine from a posterior view of the spine (FIG. 13C) and a side view of the spine (FIG. 13D). As will be appreciated from these depictions, the crossbar is mounted to a first vertebral body at a first level 1 of the spine, which the laminar clamp, or extra-laminar securement device, engages the lamina and/or spinous process at a second level 2 of the spine. As depicted the second level is adjacent the first level. As best seen in FIG. 13C, the first level 1 is the level between a lumbar vertebral body and the sacrum (see FIG. 1, L5-s5), and the second level 2 is the level between the L4 and L5 vertebral bodies. This embodiment is thus particularly well suited for attaching cephalad facet replacement components to the L5 vertebral body, as the lamina of the L5 vertebral body is generally much thinner and less robust than the lamina of the other lumbar vertebral bodies (with significantly reduced available lamina to attach a trans-laminar device thereto).

FIG. 14A illustrates a facet replacement system constructed according to an alternate embodiment wherein the laminar clamp 1440 and the crossbar mounts 1434, 1434′ are provided with optional anchoring devices 1435, 1443. The anchoring devices are configured as teeth that enable the laminar clamp 1440 and the crossbar mounts to securely (and potentially invasively) engage the lamina and/or spinous process of a spine. Further, the fixation elements 1412, 1412′ can be adapted to provide surface texturing or other features to promote bony in-growth or increase fixation strength, as described above. FIGS. 14B-D illustrate the facet replacement system of FIG. 14A implanted from various perspectives. The facet replacement device of FIG. 14 is particularly well suited to anatomical locations where trans-laminar attachment may not be an optimal solution, as well as to locations where poor laminar bone quality and/or anatomical limitations preclude the use of translaminar anchors. For example, at the L5-S1 vertebral level, the L5 lamina is generally thinner and weaker than that of other vertebral levels, Stresses on the facet joint at this level are also generally the highest of the spine and the angle of the facet joint is essentially normal to the axis of the lamina. An additional hook 1444 can be provided on the laminar clamp 1440 that enables the laminar clamp 1440 to further secure the laminar clamp 1440 to the vertebral body by engaging the vertebral arch and positioning within the vertebral foramen (see, FIGS. 2, 18, 19). As will be appreciated by those skilled in the art, the laminar anchor can be configured such that it compresses the spinous process (such as by pivoting at a location adjacent to the hook 1444, thereby compressing the spinous process between the arms of the laminar anchor).

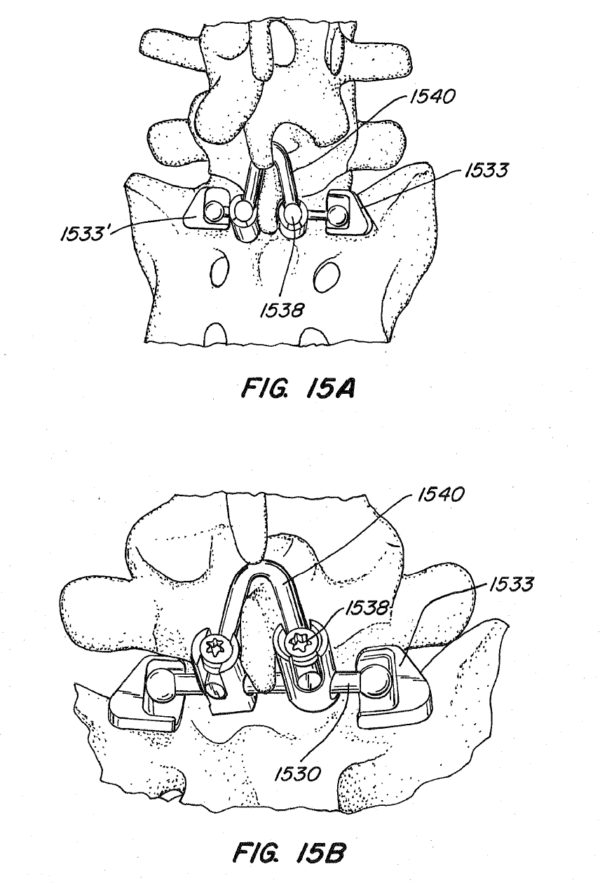

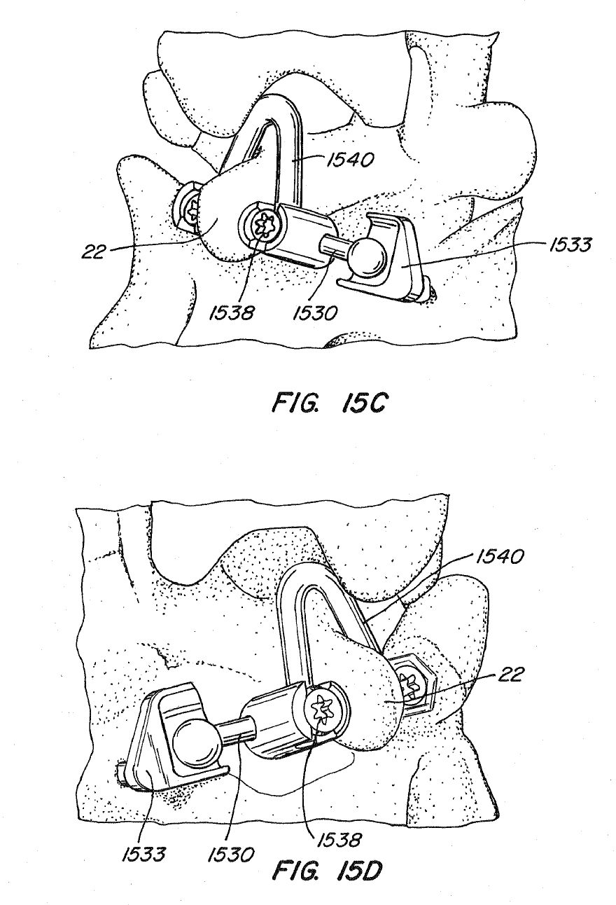

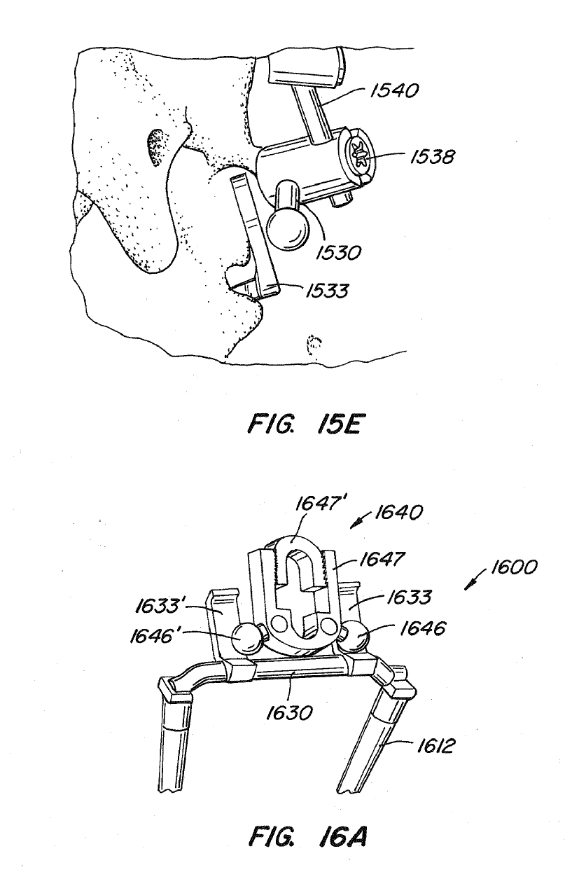

FIGS. 15A-C illustrate a facet replacement system 1500 constructed according to an alternate embodiment, from various perspectives. This embodiment of the facet replacement system is particularly well suited to anatomical locations where trans-laminar attachment may not be an optimal solution, as well as to locations where poor laminar bone quality and/or anatomical limitations preclude the use of translaminar anchors. For example, at the L5-S1 vertebral level, where approximately 50% of current intervertebral disc replacement operations occur, the L5 lamina is generally thinner and weaker than that of other vertebral levels. Stresses on the facet joint at this level are also generally the highest of the spine and the angle of the facet joint is essentially normal to the axis of the lamina. The caudad cups 1533, 1533′ are configured to rest against the sacrum. As illustrated in FIG. 15C and FIG. 15D, one portion of the cross-bar can abut against and/or contact the underside of the lamina and/or spinous process of the cephalad vertebral body, while the other side has a bearing surface that mates with a caudad cup 1533. The laminar clamp 1540 is adapted to traverse the top of the lamina/spinous process, while the crossbar traverses under the lamina/spinous process. The laminar clamp is secured to the crossbar 1530 with a pair of side screws 1538. In this embodiment, the laminar clamp and crossbar desirably compress the lamina/spinous process therebetween.

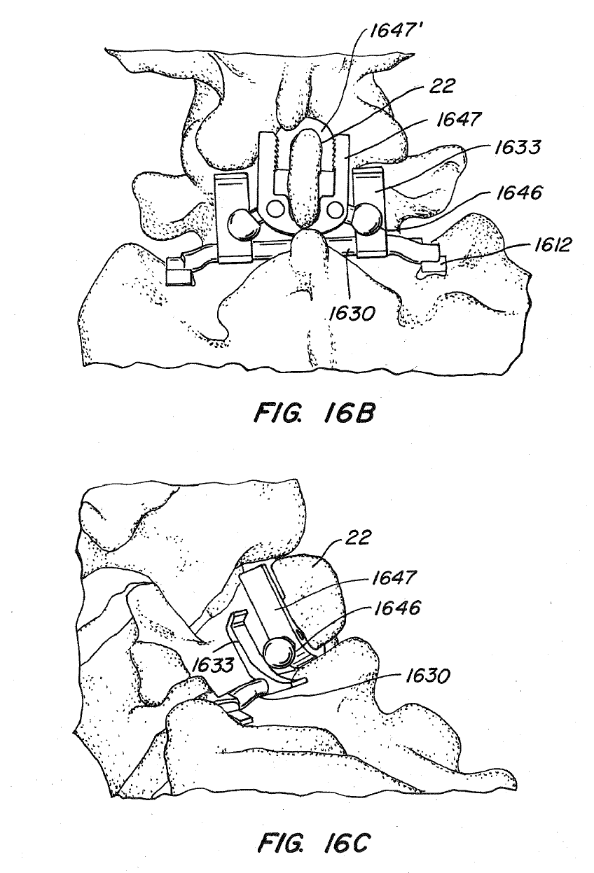

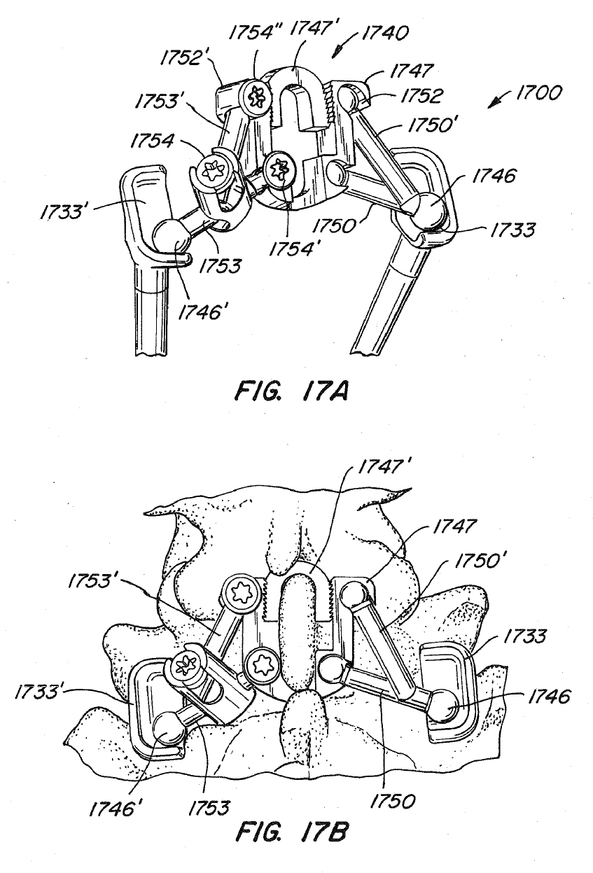

FIG. 16A illustrates a facet replacement system 1600 according to an alternate embodiment wherein the laminar clamp 1640 incorporates a modular clamp assembly. Further the cross-bar 1630 is configured to be at least partially integral with the fixation element 1612. Caudad ledges 1633, 1633′ are provided to engage bearing surfaces 1646 extending laterally from the laminar clamp 1640. Two opposing U-shaped clamps 1647, 1647′ are provided that can be ratcheted by use of, for example, an first U-shape clamp that engages the sloping teeth on a surface of a second, opposing U-shaped clamp. In operation, the ratchet mechanism permits motion in one direction only and the laminar clamp 1640 is tightened around the spinous process. FIGS. 16B-C illustrate the facet replacement system of FIG. 16A implanted from various perspectives. This embodiment of the facet replacement system 1600 is also well-suited to anatomical locations where trans-laminar attachment may not be an optimal solution, as well as to locations where poor laminar bone quality and/or anatomical limitations preclude the use of translaminar anchors. Moreover, this embodiment, as with other similar embodiments, may be utilized at various spinal locations where the natural pedicles are unsuitable for use as anchoring points, where spinal hardware and/or bone cement already occupy one or more pedicles (such as from a previous spinal fusion, other spinal procedure involving pedicular hardware, and or where a Kyphoplasty or vertebroplasy procedure has been accomplished or attempted), or where additional support for pedicle-based spinal instrumentation may be desired or required (such as where pedicle and lamina/spinous process support are both required for adequate support). As illustrated, the facet replacement system is implanted at the L5-S1 vertebral level. In this embodiment, the bearings are configured to fit within a rotatable housing.

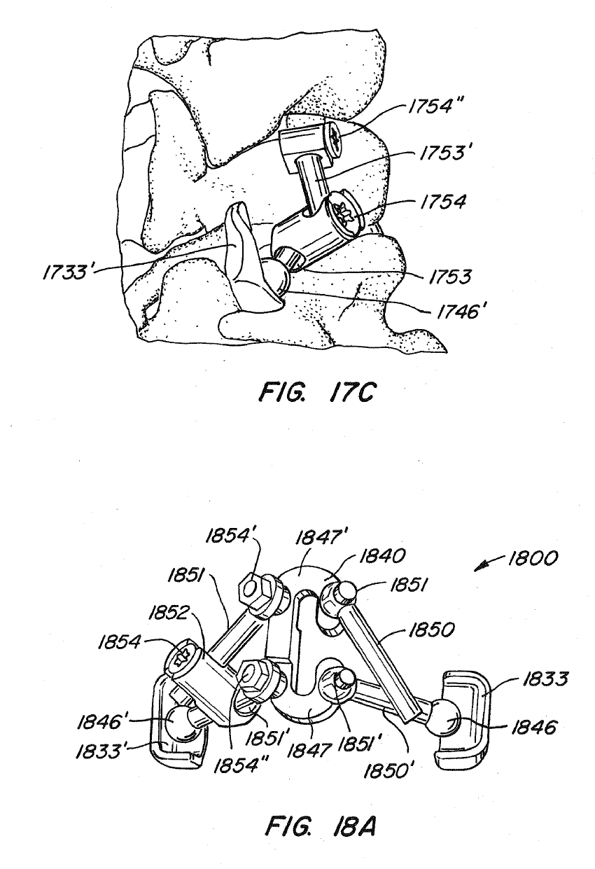

FIGS. 17A-c illustrate a facet replacement system 1700 according to yet another alternate embodiment, as well as the system 1700 implanted from various perspectives. The system has a laminar clamp 1740 formed from two opposing modular clamp assemblies. In lieu of a cross-bar (see 1630 of FIG. 16) the laminar clamp 1740 is engaged on either side by rotatable mechanisms, such as polyaxial joints, that engage a pair of cephalad balls that are adapted to engage respective bearing surfaces 1746. Two opposing U-shaped clamps 1747, 1747′ are provided that can be ratcheted by use of, for example, a first U-shape clamp that is adapted to engage the sloping teeth or detents on a surface of a second, opposing U-shaped clamp. In operation, the ratchet mechanism permits motion in one direction only and the laminar clamp 1740 is tightened around the spinous process. To release the laminar clamp (e.g., to provide a looser fit), the inner clamp is pushed slightly toward the opposing clamp while compressing the side members of the U-shaped clamp. Thereafter, the inner clamp is withdrawn to the desired position, or removed entirely.

As illustrated, on one side, the bearing surface has a cross-bar 1750, 1750′ that extends from the bearing surface 1746 to engage the externally positioned U-shaped clamp 1747′. As depicted herein, the surface of the clamp nearest the caudad cup 1733′ is adapted, e.g. forming a socket 1752, to receive a rounded ball end of the cross-bar. On the other side of the device, one or more joints 1152′ are provided into which the cross-bars 1753, 1753′ can be locked using a locking mechanism 1754, 1754′, 1754″. Providing more than one cross-bar on either side of the laminar clamp 1740 with poly-axial connectors enables the device to achieve a greater degree of flexibility.

FIGS. 17B-c illustrate the facet replacement system of FIG. 17A implanted from various perspectives. This embodiment of the facet replacement system 1700 is also well-suited to anatomical locations where trans-laminar attachment may not be an optimal solution, as well as to locations where poor laminar bone quality and/or anatomical limitations preclude the use of translaminar anchors. As illustrated, the facet replacement system is implanted at the L5-S1 vertebral level.

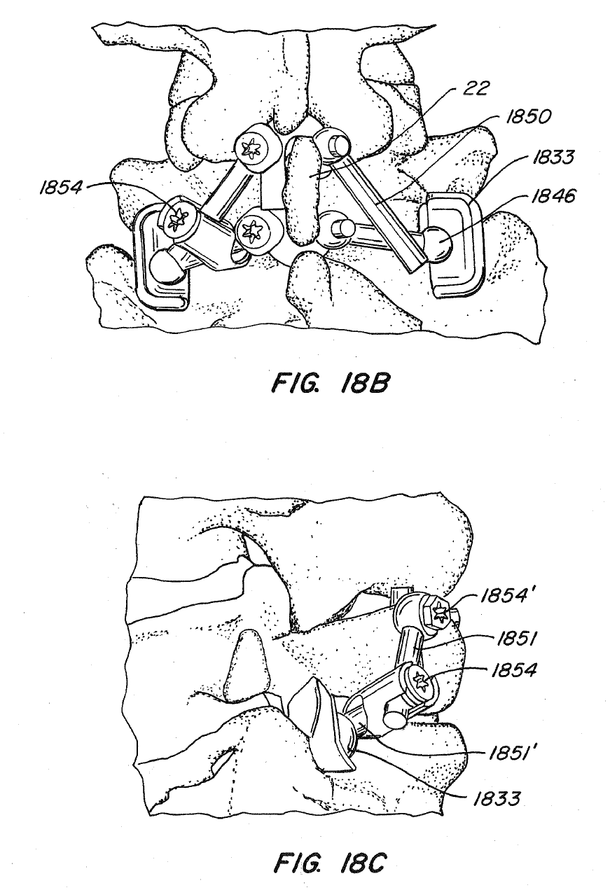

FIG. 18A illustrates a facet replacement system according to an alternate embodiment wherein the laminar/spinous process clamp is an adjustable C-clamp. The system 1800 has a clamp 1840 which can be formed from two opposing modular clamp assemblies. In lieu of a cross-bar (see 1630 of FIG. 16) the clamp 1840 is engaged on either side by an adjustable mechanism carrying a pair of cephalad balls at their distal ends. Caudad cups 1833, 1833′ are provided on the caudad components of the facet replacement system to engage respective bearing surfaces 1846 of the cephalad balls. Two opposing clamps 1847, 1847′ are provided that can be ratcheted into a C-shaped clamp. For example, a first clamp that engages the sloping teeth on a surface of a second, opposing clamp; when combined the clamps form a C-shape. If fully ratcheted, the open end of the clamp can meet (if desired). In operation, the ratchet mechanism permits motion in one direction only and the laminar clamp 1840 is tightened around the spinous process. As illustrated, on one side, the bearing surface has a first set of cross-bars 1850, 1851 that extend from the bearing surfaces 1846, 1846′ to engage the clamp 1847′, at a cephalad location, and a second set of cross-bars 1850′, 1851′ to engage the clamp at a caudad location. As depicted herein, the surfaces of the clamp are adapted, e.g. forming a socket, to receive rounded ball ends of the cross-bars. On the other side of the device, one or more joints 1852 are provided into which the cross-bars 1851, 1851′ can be locked using a locking mechanism 1854, 1854′, 1854″. Providing more than one cross-bar on either side of the laminar clamp 1840, enables the device to achieve a greater degree of flexibility and strength.

FIGS. 18B-c illustrate the facet replacement system of FIG. 18A implanted from various perspectives. As with previous embodiments, this embodiment of the facet replacement system 1800 is also well-suited to anatomical locations where trans-laminar attachment may not be an optimal solution, as well as to locations where poor laminar bone quality and/or anatomical limitations preclude the use of translaminar anchors. As illustrated, the facet replacement system is implanted at the L5-S1 vertebral level.

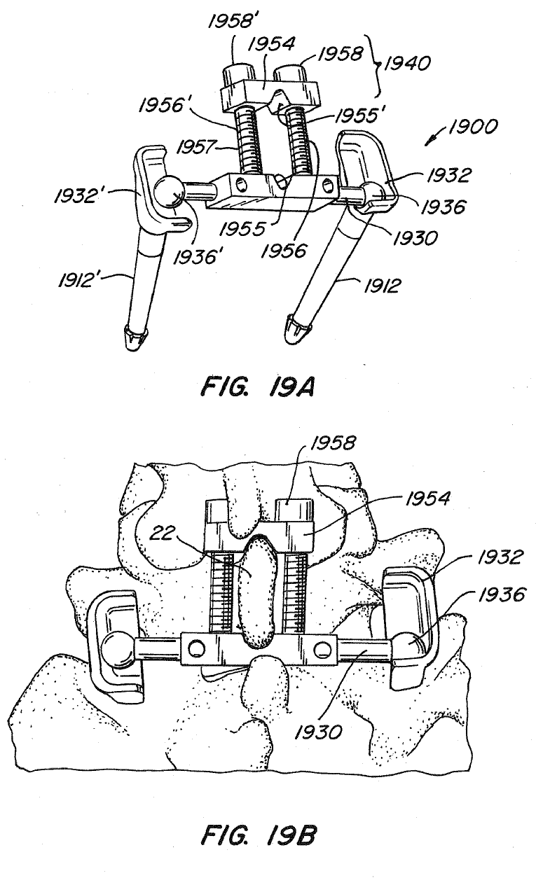

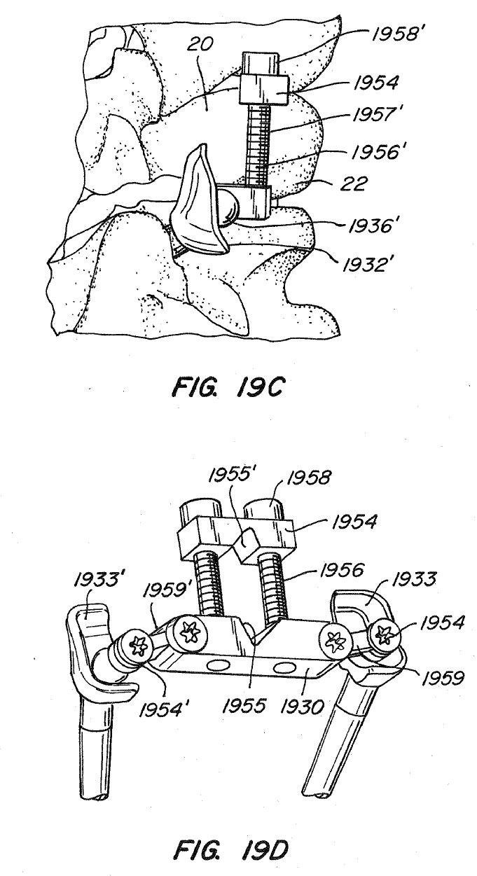

Turning now to, FIG. 19A a facet replacement system 1900 according to an alternate embodiment is illustrated wherein the laminar clamp 1940 incorporates adjustable rods and the cross-bar is adjustable in length (e.g., across a midline of the spine, formed by the sagittal plane 54, while lying in an axial plane 52). Fixation elements 1912, 1912′ are provided having caudad cups 1932, 1932′ for receiving a corresponding bearing surface 1936, 1936′ of a cross-bar 1930, which can be adapted to provide a divot 1955 to receive a portion of the lower curved surface of the spinous process and can further be adapted to provide an adjustable length. The fixation elements 1912, 1912′ can be adapted to provide surface texturing or other features to promote bony in-growth, as described above with respect to other embodiments. Further, the laminar clamp 1940 has a bearing clamp 1954 adapted to engage an upper surface of a spinous process 22, such as by providing a divot 1955′ sized to receive the spinous process. The bearing clamp 1954 which engages a pair of vertical, adjustable rods 1956, 1956′ which can be provided with markings 1957 to, for example, enable the surgeon to assess the length of the implanted device. The length of the rods can be adjusted as desired. Additional internally threaded caps 1958, 1958′ can be provided to engage the end of the adjustable rods 1956 after the rod passes through an aperture in the bearing clamp 1954. FIGS. 19B-C illustrate the facet replacement system of FIG. 19A implanted from various the posterior view and a lateral view. If desired, the vertical, adjustable rods 1956, 1956′ can be sized and positioned to provide a lateral clamping force about the sides of the spinous process and/or lamina as well.

The facet replacement device of FIG. 19A is also well suited for anatomical locations where trans-laminar attachment may not be an optimal solution, as well as to locations where poor laminar bone quality and/or anatomical limitations preclude the use of translaminar anchors for the reasons discussed above. FIG. 19D illustrates a facet replacement system according to an alternate embodiment wherein the crossbar 1930 has a pair of jointed rods 1959, 1959′ which are secured to the internally threaded bearings 1936, 1936′ by anchors 1954, 1954′. The jointed rods 1959, 1959′ enable the position of the crossbar 1930 to pivot in relation to the location of the bearings 1936 within the caudad cups 1933, 1933′. FIG. 19E illustrates the facet replacement system of FIG. 19D implanted. As can be appreciated from this illustration, the implantation of the caudad cups 1933, 1933′ in relation to each other within a plane is such that the cup on the patient's right is higher than the cup on the patient's left. The crossbar 1930 is nonetheless positioned across the spine in relation to the lamina and or spinous process between the cups. As a result of the jointed rods, the cross-bar member can remain in a neutral position while the bearings are optimally positioned within the caudad cups by angling the bearings relative to the axis of the crossbar to take into account the positioning of the caudad cups. The solid bar fits into the lower cross-arm and allows a change in shaft length. The locks, such as clamp 1954 enable the device to be secured, if desired, into a particular configuration. Alternatively, the clamp can act as a pivot point, allowing the device to dynamically adjust to the patient's anatomy in situ during normal day-to-day activities.

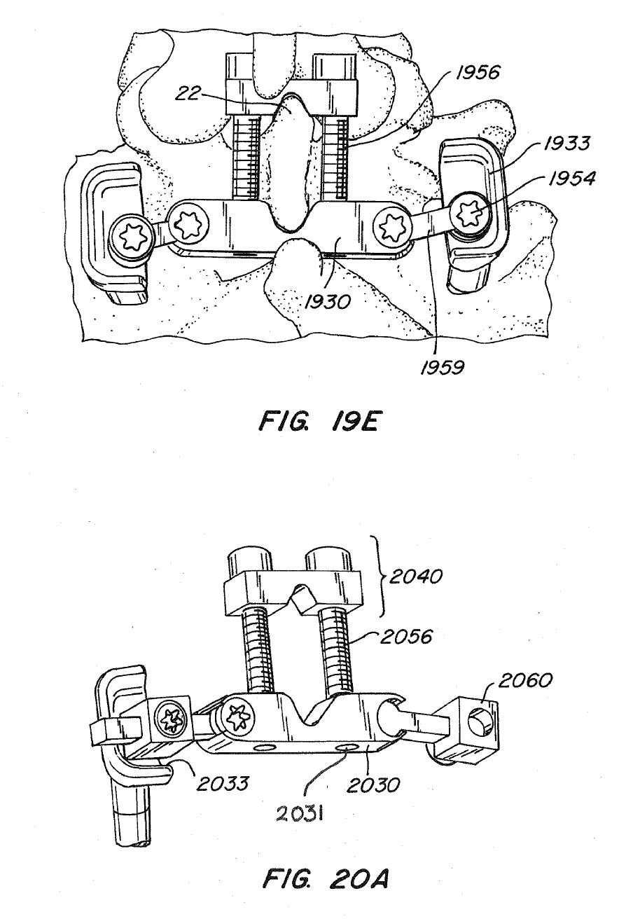

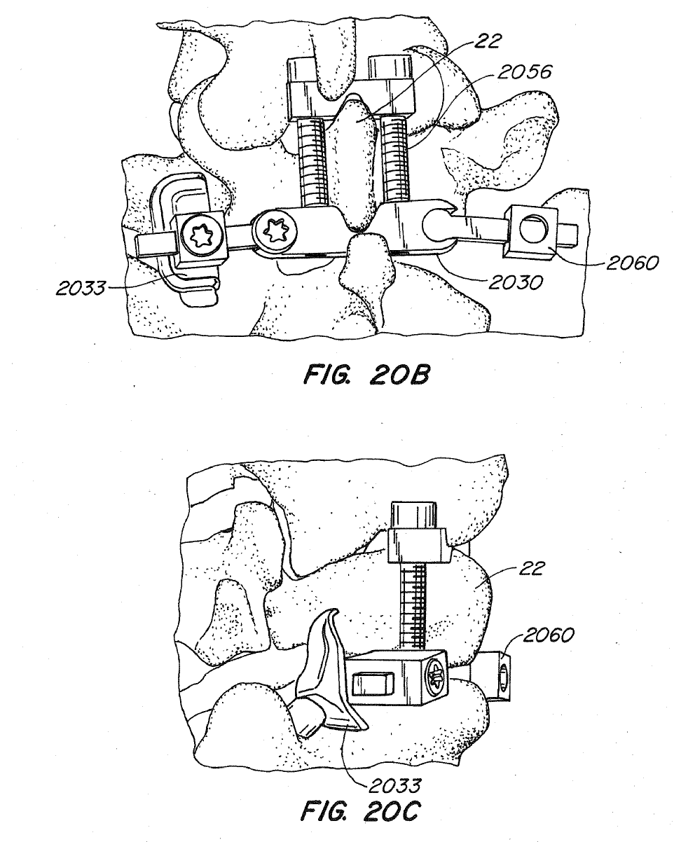

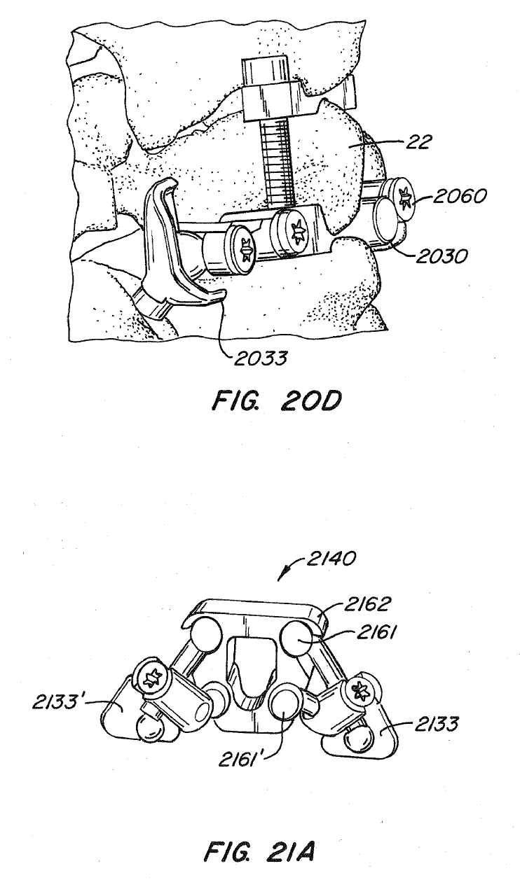

FIG. 20A illustrates a facet replacement system according to an alternate embodiment wherein the laminar clamp 2040 has adjustable rods 2056 and at least one section of the cross-arm 2030 is anchorable directly to (and/or into) the vertebral body, lamina and/or spinous process through the use of a set screw or pin (not shown) extending upward through an opening 2031 in the cross-arm 2030 and against and/or into the targeted bony structure. The ends of the crossbar 2030 can be configured to carry one or more bearing surfaces (not shown) within the respective caudad cup 2033. FIGS. 20B-D illustrate the facet replacement system of FIG. 20A implanted from posterior, lateral and perspective view. The configuration of this embodiment enables the cross-arm to be moved relative to the caudad cup 2033. Additionally, the cross-arm 2030 can rotate relative to the vertebral body and/or relative to the caudad bar.

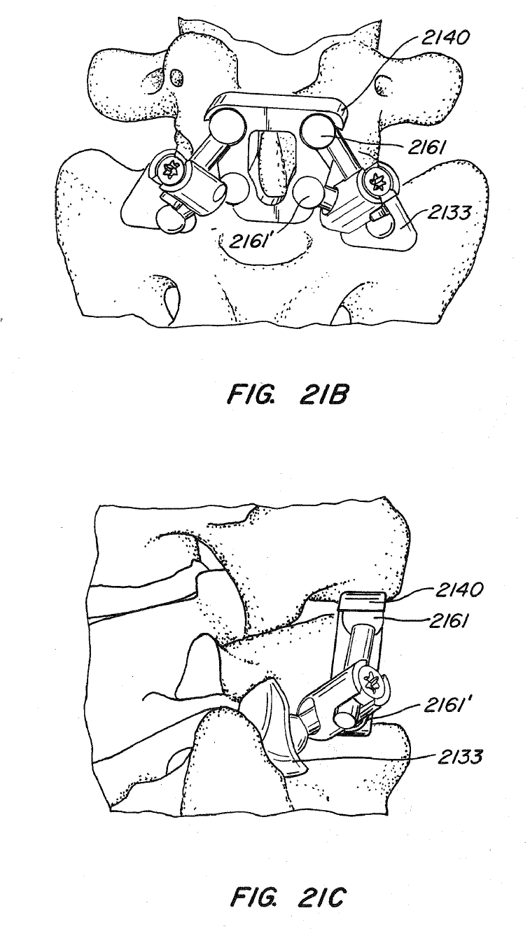

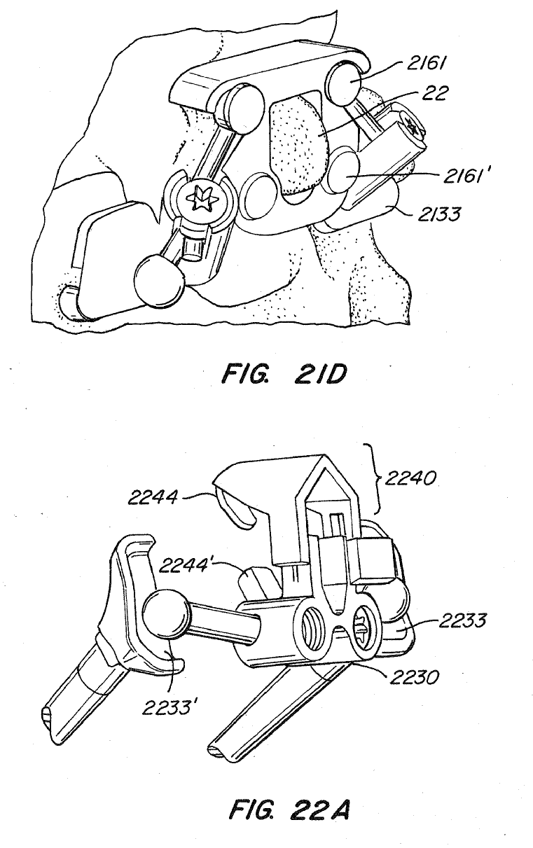

FIG. 21A illustrates a facet replacement system according to an alternate embodiment wherein the laminar clamp 2140 has a jointed linking mechanism 2162 that enables the laminar clamp to assume a range of positions when positioning within a spine. The jointed linking mechanism has two bearing surfaces with a lockable joint. Two of the bearing surfaces moveably engage the laminar clamp. The third bearing surface (the cephalad bearing surface) moveably engages (or articulates with) the caudad cup 2133. The lock controls the angle between the rods presenting the bearings to the respective clamp or cup locations thus enabling the device to be positioned within the spine, while securing the relationship between the bearing surfaces. The lock does not, however, prevent the cephalad bearing surfaces from moving in relation to the caudad cup. FIGS. 21B-D illustrate the facet replacement system of FIG. 21A implanted from the posterior, lateral and perspective views. The use of the device of FIG. 21 enables the physician to introduce two or more pieces during implantation which are then assembled into the final device.

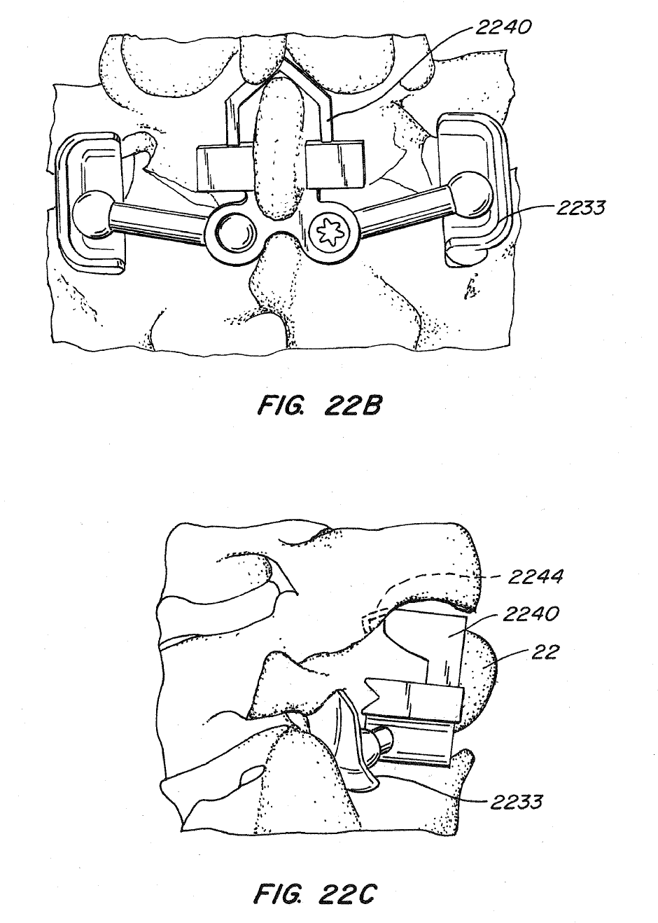

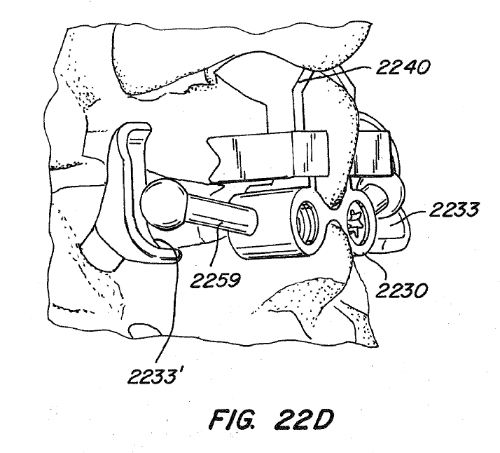

FIG. 22A illustrates a facet replacement system according to an alternate embodiment wherein the laminar clamp 2240 is a modular clamp having an upper clamp section and a lower clamp section adapted to engage each other snugly around the lamina and/or spinous process when deployed. An anterior facing hook 2244 is provided on the laminar clamp 2240 for engaging part of the vertebral body. The hook 2244 enables the laminar clamp 2240 to further secure the laminar clamp 2240 to the vertebral body by engaging the vertebral arch and positioning within the vertebral foramen (see, FIG. 2, 18, 19). A jointed rod and bearing element is also provided that enables the laminar clamp 2240 to be adapted to articulate with the caudad cups 2233. This flexibility in adapting the bearing surfaces, enables the device to be positioned within the spine such that the operation of the device is optimized relative to the native or resected anatomy. FIGS. 22B-D illustrate the facet replacement system of FIG. 22A implanted from the posterior, side and perspective views. In operation, the device can clamp onto the lamina and/or spinous process. An upward facing, opposing clamp 2244′ can be adapted to extend from the crossbar 2230 section to further engage the vertebral body. Further, a solid crossbar section can be adapted to engage the laminar clamp 2240. Lockable jointed rods 2259 can be provided which are adapted to extend from the crossbar 2230 to engage the caudad cups 2233, 2233′; thus allowing further configurability and flexibility to the device.

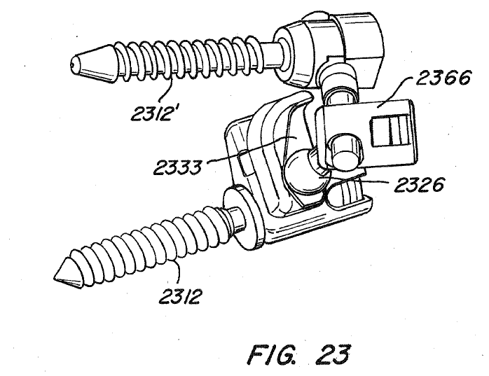

FIG. 23 is a side view of one side of a portion of a facet replacement system illustrating the linking feature. As illustrated in this embodiment, which includes linking and jointing features of some of the embodiments described above, components can be linked together such that the components are inflexibly or flexibly linked to allow articulation between components (e.g., bearing surfaces). Alternatively, the components can be linked to allow movement and/or displacement between the components. If desired, at least one end of the linking device can comprise a polyaxial type connection to connect to one or more components of the facet replacement system. In alternative embodiments, the link can be adapted to pass through one or more openings formed through various components of the system. In this embodiment, two fixation mechanisms are provided 2312, 2312′. The fixation elements are, as illustrated, threaded to enable the threaded element to anchor within a target section of the spine, such as the sacrum or the pedicles of other vertebral bodies. A bearing forming the cephalad joint surface 2326 fits within a caudad cup 2333. The bearing is adapted to engage a lockable pivot mechanism 2366 which enables customization of the location of the cephalad bearing within the caudad cup.

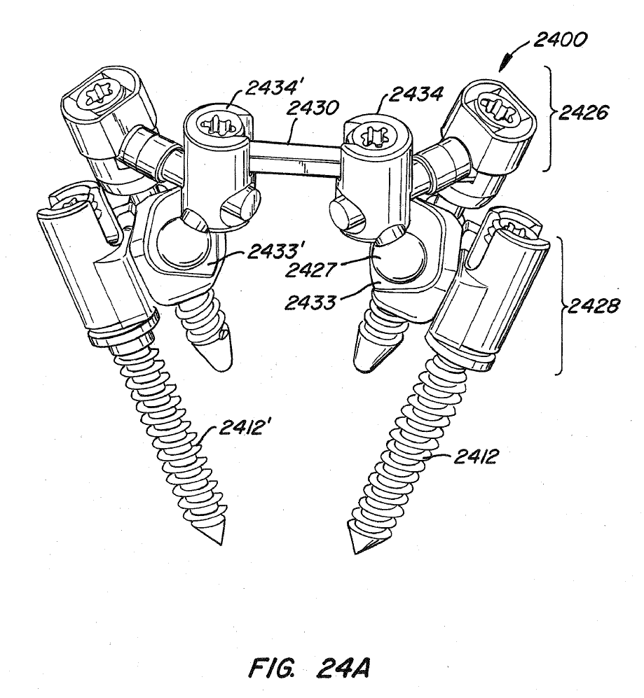

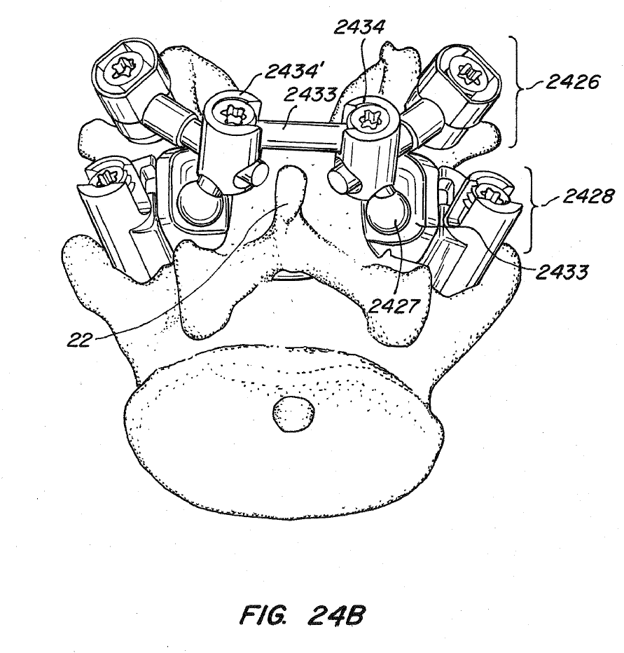

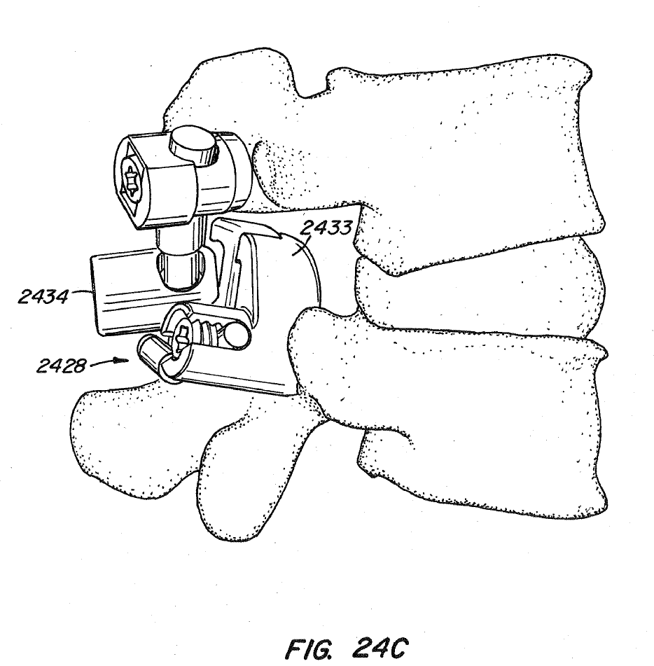

FIGS. 24A-C are various views of the facet replacement system of FIG. 23. The device 2400 is an assembled configurable and adaptable spinal restoration device. This embodiment illustrates how the various components of the device can be selected and configured to accommodate an individual's anatomy.

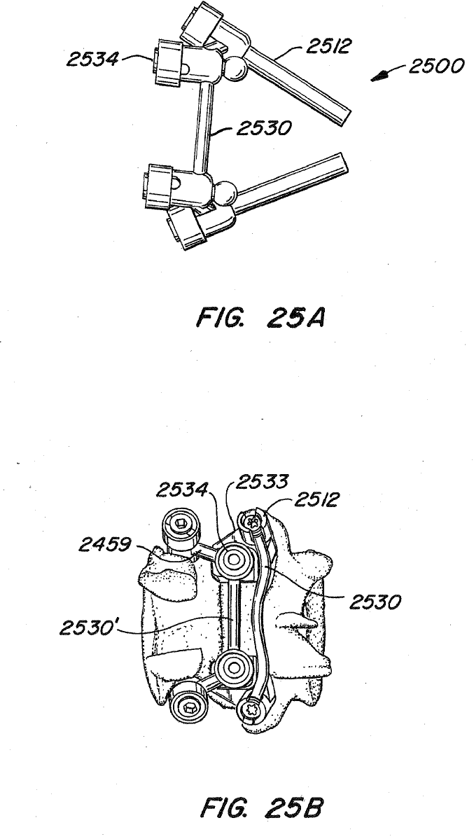

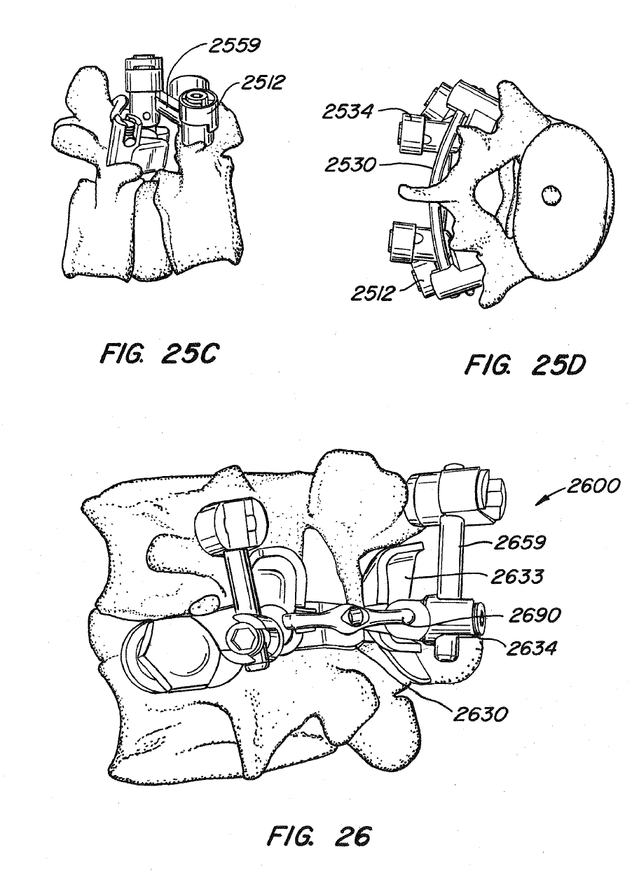

FIG. 25A is a top view of a cephalad interconnection device constructed according to the various teaching of the present invention; FIG. 25B-D illustrate the device of FIG. 25A implanted from a posterior view, superior view and a lateral view, in conjunction with a caudad interconnection device incorporating a caudad crossbar. Many of the components are similar to those in previous embodiments including, for example, the use of fixation elements 2512. In this embodiment, two crossbars 2530, 2530′ are provided. The first crossbar 2530 is adapted to connect to a cephalad anchoring system having two anchoring devices positioned on the cephalad (or upper) vertebral body. The second crossbar 2530′ is positioned below the first crossbar and is adapted to connect two caudad devices 2528.

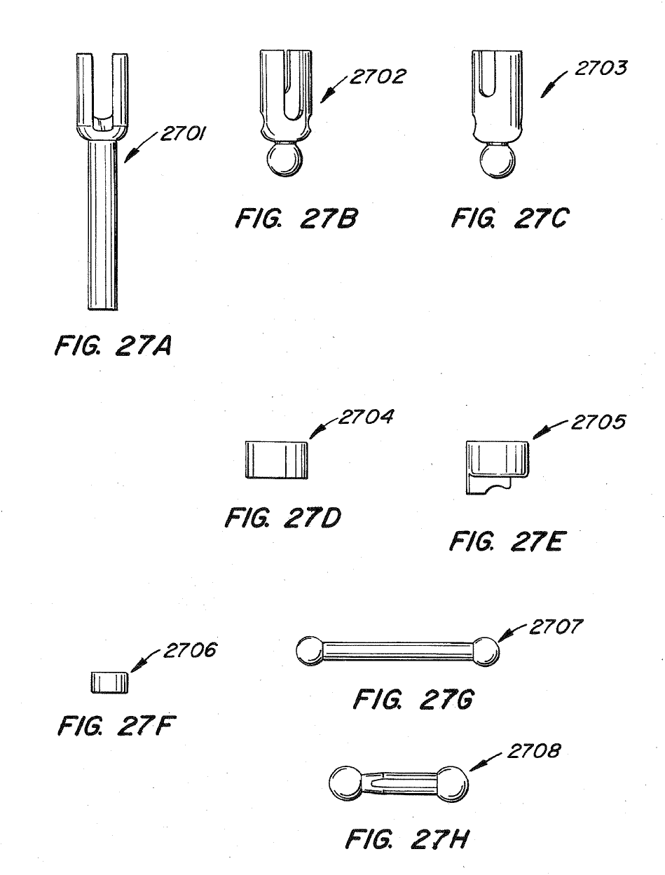

FIGS. 27A-H illustrate the components of a translaminar facet arthroplasty cephalad construct system, such as described in FIGS. 25A-D. FIG. 27A illustrates a pedicle screw or stem 2701; FIG. 27B illustrates a left housing 2702; FIG. 27C illustrates a right housing 2703; FIG. 27D illustrates a housing cap 2704; FIG. 27E illustrates a pedicle screw cap 2705; FIG. 27F illustrates a set screw 2706; FIG. 27G illustrates a cross-bar press fit assembly 2707; and FIG. 27H illustrates a cephalad arm press fit assembly 2708. The pedicle screw 2701 has an elongated shaft with a notched tubular housing adapted to receive a bearing within the housing and to engage a crossbar associated with the bearing through the notch. The left housing 2702 and the right housing 2703 are configured to provide a rounded bearing at one end and a notched tubular housing at the opposing end. The tubular housing is adapted to receive a bearing within the housing and to engage a crossbar associated with the bearing through the notch. The housing cap 2704 and the pedicle screw cap 2705 can be adapted to fit within the open end of the elongated shaft to secure a bearing within the shaft. The set screw 2706 can further be adapted to fit within either of the housing cap 2704 or pedicle screw cap 2705 to further secure the cap to, for example, the pedicle screw. The crossbar press fit assembly 2707 is adapted to engage the pedicle screw or the left or right housing and can be further configured, for example, to have a diameter of approximately 4 mm with two bearings on either end with a 5/16″ diameter. The cephalad arm press fit assembly 2708 can also be configured to engage the pedicle screw or housings and to have a 5 mm diameter that transitions to a 4.5 mm diameter bar; also with two 5.16″ diameter bearings on either end. Further the cephalad arm press fit assembly can further be modularized such that the shaft is comprised of more than one piece. Additionally, the device could have a single piece construct as will be appreciated by those skilled in the art.

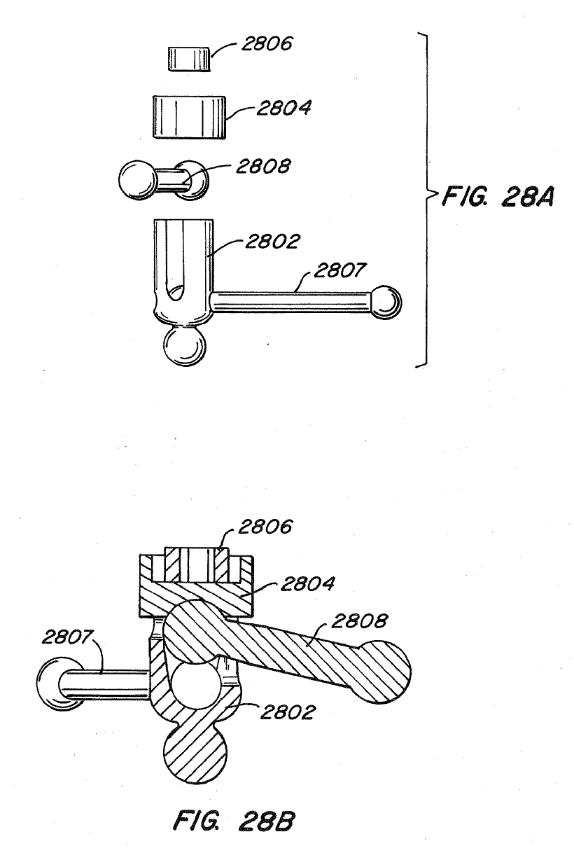

FIGS. 28A-B illustrates components of the translaminar facet arthroplasty cephalad construct system shown in FIG. 27 in construction. As shown in FIG. 28B, the crossbar fit assembly 2807 slides into the left housing 2802 such that the crossbar of the fit assembly 2807 forms an angle, such as a right angle as depicted, with the elongated shaft of the housing 2802. Thereafter, the cephalad press fit assembly 2808 slides within the housing 2802 such that the assembly 2808 is positioned over the assembly 2807. The crossbar of the press fit assembly 2808 slides within the housing such that the assembly forms an angle with respect to the shaft. While the crossbar fit assembly 2802 and the press fit assembly 2808 can be configured to lie in parallel planes, as can be seen from the illustration, each of the assemblies will extend from the shaft of the housing 2802 such that from a superior view, an angle is formed between the assemblies with the shaft as a focal point. The configuration can be maintained in place with housing cap 2804.

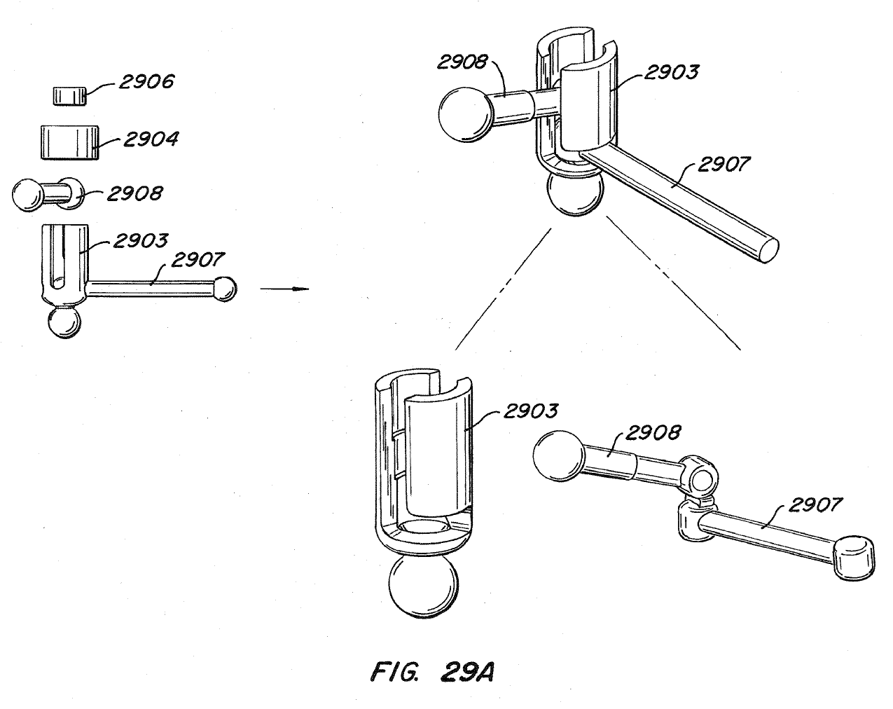

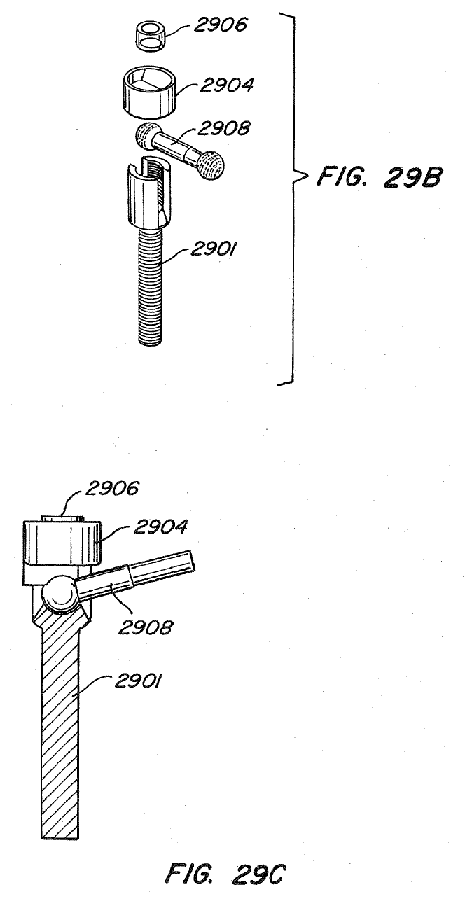

FIGS. 29A-C illustrates a facet arthroplasty system cephalad construct according to an alternate embodiment employing the components of FIG. 27. As can be seen in FIG. 29A, the notch in the shaft is configured to enable the assembly 2907 to slide down into the shaft and then be turned along a notch that is perpendicular to the access notch (thus forming an “L” shaped notch). As illustrated in FIG. 29B-C, once the assembly is positioned within the shaft, the bottom can be configured such that the arm extends from the housing at an angle other than 90°. The shaft of the fit assembly 2907 can further be adapted to be telescoping such that overlapping sections are provided that can slide inward or outward to lengthen or shorten the shaft.

FIGS. 30A-C illustrate a facet arthroplasty cephalad construct system according to an alternate embodiment. The system includes a circular flange housing into which a set screw is placed to anchor the system together. The set screw locks the cephalad arm and the outside lock locks the crossbar.

FIGS. 31A-B illustrate another component of a construct system suitable for use with a disc-facet arthroplasty system. A malleable or pre-formed plate 3100 is provided that is adapted to secure a cephalad bearing to the bone. The cap structure 3101 fits on one side of a lamina, and can form a cephalad bearing surface, if desired. A rod can be inserted through the cap on one side of the lamina and through an aperture 3103 in the arm on the other side of the lamina (the rod can comprise a trans-laminar cephalad fixation mechanism as previously-described). The arm 3102 secures the cephalad bearing to the lamina, which may be augmented using laminar screws through one or more of the remaining apertures in the plate. This system may be particularly well suited for use in conjunction with the various trans-laminar cephalad anchoring systems, such as the various systems described in FIGS. 4-12, and may be utilized, if desired, to link a pair of translaminar anchoring devices that are passing through the same lamina and/or spinous process.

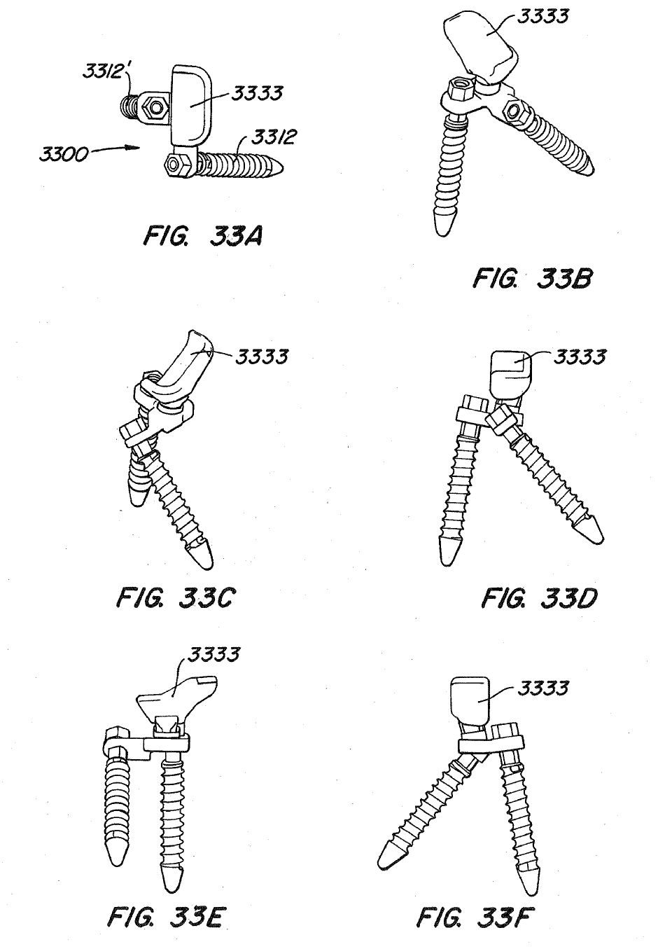

FIGS. 33A-F illustrate various views of a fixation device suitable for use at a sacral connection for a caudad cup 3333. As would be appreciated by those skilled in the art, the sacrum may not be of the strongest bone quality and/or any spinal implant in the sacrum will likely experience the highest compressive and bending loads in the spine. Accordingly, securing mechanical devices to the sacrum presents an additional challenge. Once mechanism for overcoming this challenge is to provide a dual fixation caudad cup design 3300, such as that depicted in FIG. 33A-F. The dual fixation device 3300 enables the caudad cup 3333 to be secured from two angles by the use of two fixation elements 3312, 3312′. This design is able to better secure the caudad cup to the sacrum. In the disclosed embodiment, the device 3300 desirably secures each of the caudad cups 3333 to the sacrum with one fixation device (i.e., a fixation screw), and to the sacral ala with a second fixation device (i.e., a second fixation screw).

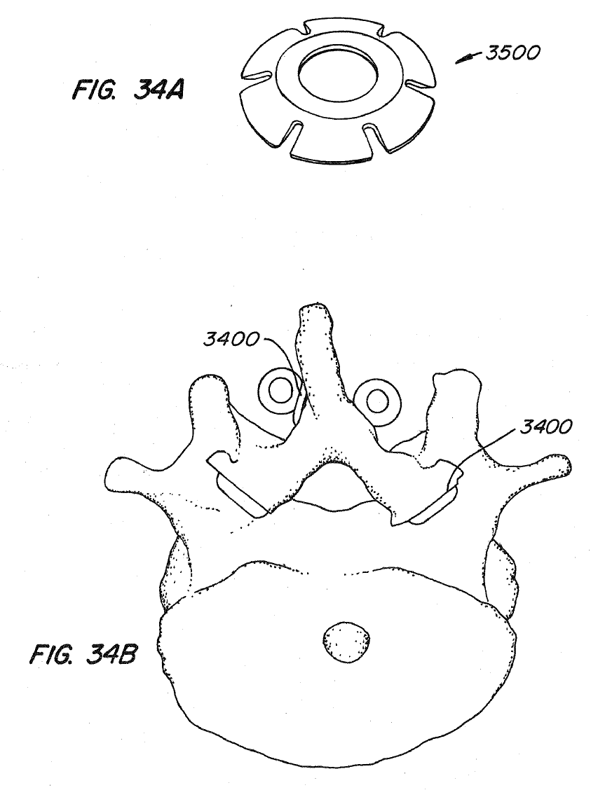

FIGS. 34A-B illustrate a cephalad translaminar fixation system incorporating the use of a spring washer 3400. The spring washer is configured in a petal design to allow the washer edges to conform to the irregular bone surface. The springiness of the washer desirably creates tension which better secures the device to the bone. The washer can be used at any location where a device is adapted to engage bone and is suitable for use with any of the embodiments disclosed herein. The washer may also incorporate a textured or bony in-growth surface to facilitate bony fixation.

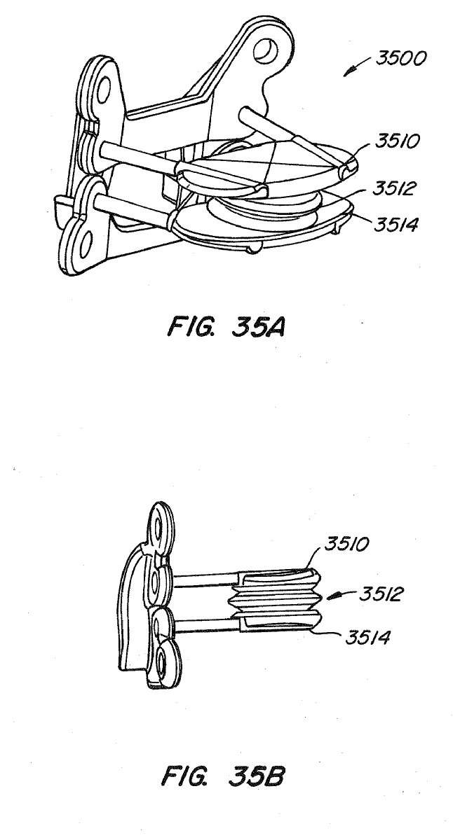

FIGS. 35A-B illustrate a disc replacement device 3500 in combination with a facet replacement component. The device is adapted to attach to a portion of the facet replacement device. In turn, the facet replacement device engages a portion of each of the vertebral bodies (although, in alternative embodiments, the facet replacement device may be solely anchored to the disc replacement device, and the disc replacement device may or may not be anchored to the surrounding vertebral bodies). The disc component of the device can be any artificial device capable of at least partially restoring the natural motion of the intervertebral disc. The disc can be an articulating disc, a cushion disk and a spring-based disc. Various disc replacement devices are described in U.S. Pat. No. 5,071,437 to Stefee et al. for Artificial Disc; U.S. Pat. No. 6,113,637 to Gill et al. for Artificial Intervertebral Joint Permitting Translation and Rotational Motion; U.S. Pat. No. 6,001,130 to Bryan et al. for Human Spinal Disc Prosthesis with Hinges; U.S. Pat. No. 4,759,769 to Hedman et al. for Artificial Spinal Disc; U.S. Pat. No. 5,527,312 to Ray et al. for Facet Screw Anchor; U.S. Pat. No. 5,824,094 to Ray et al. for Spinal Disc; U.S. Pat. No. 5,401,269 to Buttner-Janz for Intervertebral Disc Endoprosthesis; 5,824,094 to Serhan et al. for Spinal Disc; 5,556,431 to Buttner-Janz for Intervertebral Disc Endoprosthesis; U.S. Pat. No. 5,674,296 to Bryan et al. for Human Spinal Disc Prosthesis; and U.S. Patent Pub US2005/0055096 A1 to Serhan et al. for Functional Spinal Unit Prosthetic. The articulating motion disc can have a three piece design 3510, 3512, 3514 with two endplates 3510, 3514 and a core 3512. Each of the plates can be provided with one concave surface adapted to receive a convex surface presented by the core; thus forming a ball-and-socket joint.

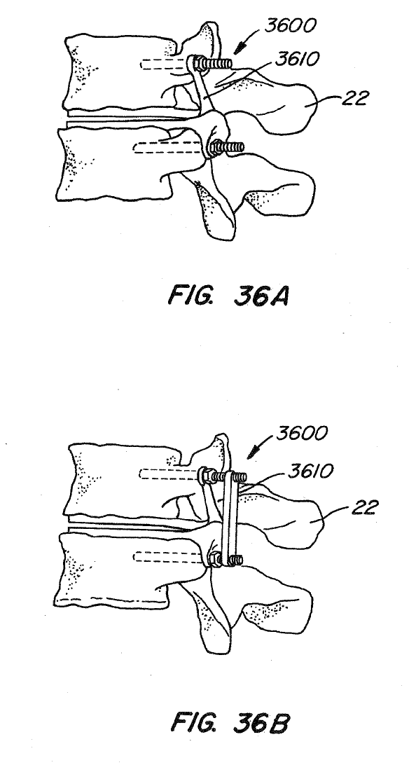

FIGS. 36A-B illustrates a disc replacement device 3600 according to an alternative embodiment with a facet replacement component and artificial linkages 3610 between the vertebral bodies. In this embodiment, the disc replacement device engages both a portion of the facet replacement device as well as a portion of the vertebral body. Moreover, this embodiment includes a trans-vertebral link between the components of the facet replacement device that can be created from a variety of materials including, for example, titanium, stainless steel, or radiolucent polymer materials such as polyether ether ketone (PEEK™) provided by Victrex PLC (United Kingdom). The trans-vertebral link may or may not be rigid See, for example, U.S. Patent Pub. US2005/0033434 A1 to Berry for Posterior Elements Motion Restoring Device. Attachment between the facet prosthesis and disc not only reduces or obviates the opportunity for migration of the artificial disc replacement, it also reinforces and/or augments the anchoring of the facet replacement component to the vertebral body, as well as preventing subsidence of the artificial disc replacement into the respective upper or lower endplate of the treated vertebral bodies, Moreover, such attachment allows the attachment mechanism to be utilized in a minimally invasive fashion to reposition the artificial disc replacement within the disc space (anterior/posterior and/or laterally, or a combination thereof),

In addition, attachment between facet prosthesis and disc can alter the loading on the artificial disc replacement, if desired. For example, where the artificial disc is failing in some mode of operation (such as during anterior loading of the disc), repositioning of the disc replacement in a more anterior location may alter loading of the disc to a more posterior direction, thereby extending the life of the disc replacement before removal, replacement and/or repair (and subsequent surgical intervention) is required. In a similar manner, utilizing non-symmetrical connections between the anterior and poster vertebral bodies, can allow you to preferentially load the disc replacement prosthesis in a non-symmetrical manner, or account for anatomical deformities that preclude or prevent the insertion of a symmetrical (i.e. -standard) spinal joint replacement device.

FIG. 26 is a perspective view of an implanted system that incorporates an artificial disc replacement (not shown) and a facet arthroplasty system. The system 2600 can be modularized, using the features described above, or can be integrally formed such that the components essential or necessary for completeness are provided enabling the device to operate in a unified manner. Alternatively, the system can be formed such that the components are interconnected in a seamless manner. This embodiment is constructed to enable the device to be deployed using a percutaneous procedure. The bearings are inverted which enables a less-invasive approach. The central cephalad link 2690, in combination with the central caudad link 2630, enables the components to be secured or linked together during installation and then removed. Articulation can also be limited or prevented, if desired. Connection mechanisms can also be provided between the linkage and the artificial disc; such mechanisms can further serve to augment the stability and long-term viability of the artificial disc replacement and/or the facet replacement device. The central caudad link 2630 may further comprises an anteriorly extending arm (not shown) that travels along an endplate of the vertebral body through an opening formed in the artificial disc replacement and extending further along the endplate. The arm can be adapted to further distribute loading of the disc on the endplate, reducing and/or eliminating subsidence of the disc replacement into and/or through the vertebral endplate. Distribution of loading occurs as a result of distributing the effect of force over a larger surface area. Various embodiments of the arm can comprise a flattened or hemi-circular cross-section, with the flattened section positioned toward the endplate.

The invention includes systems that include a single functional spinal unit joint replacement system. The devices, systems and methods provided herein reduce and/or eliminate replacement, repair and/or displacement of the artificial disc replacement device relative to the vertebral bodies during the life of the implantation. By linking disc replacement to the facet replacement, the added benefit of reducing or redistributing the loading of the spinal anchors (pedicle, lamina, spinous process and/or a combination thereof) can be achieved.

In some embodiments it may be desirable to incorporate artificial ligaments between the articulating arms and/or the treated vertebral bodies. Additionally, in some embodiments it could be desireable to incorporate a flexible capsule around some or all of the facet/articulating joint or its surfaces. Alternatively, the facet replacement device can be adapted to incorporate multiple attachment points (apertures, holes, hooks, etc.) for attachment of existing ligaments, tendons and/or other soft or hard tissues at the conclusion of the surgical procedure to promote healing and further stabilization of the affected levels.

The devices and components disclosed herein can be formed of a variety of materials, as would be known in the art. For example, where the devices have bearing surfaces (i.e. surfaces that contact another surface), the surfaces may be formed from biocompatible metals such as cobalt chromium steel, surgical steel, titanium, titanium alloys (such as Nitinol), tantalum, tantalum alloys, aluminum, etc. Suitable ceramics, including pyrolytic carbon, and other suitable biocompatible materials known in the art can also be used. Suitable polymers include polyesters, aromatic esters such as polyalkylene terephthalates, polyamides, polyalkenes, poly(vinyl) fluoride, PTFE, polyarylethyl ketone, and other materials that would be known to those of skill in the art. Various alternative embodiments of the spinal devices and/or components could comprise a flexible polymer section (such as a biocompatible polymer) that is rigidly or semi rigidly fixed such that the polymer flexes or articulates to allow the vertebral bodies to articulate relative to one another.

Various embodiments of the present invention relate to a total spine joint replacement system comprising a modular facet joint replacement in combination with an artificial spinal disc replacement device. Virtually all of the various embodiments disclosed here could be utilized, in various ways, in combination with artificial disc replacement devices, as well as nucleus repair systems and replacement devices, interbody spacers, dynamic stabilization devices, articulating rod and screw systems, posterior ligament or annular repair and/or augmentation devices, interspinous spacers, facet resurfacing devices, and the like, with varying utility.

Various embodiments of the present invention desirably link the facet replacement prosthesis with the artificial disc replacement prosthesis in some manner. This link can be integral, such that the two components are “hard linked” together (either inflexibly, or flexibly—to allow and/or disallow articulation between components), or the components can be “soft linked” together, to allow movement and/or displacement between the components to some desired limit. If desired, at least one end of the linking device can comprise a polyaxial-type connection to connect to one or components of the facet replacement prosthesis. In alternate embodiments, the link may similarly pass through one or more openings formed through the various facet replacement components.

Desirably, the limitations and disadvantages inherent with many prior art facet replacement systems, as well as many artificial disc replacement systems, can be reduced, minimized and/or eliminated by the combination of such systems into a single, functional spinal unit joint replacement system. For example, the opportunity for the disc replacement to migrate and/or displace relative to the vertebral bodies during the life of the implantation may be reduced and/or eliminated by linking the disc replacement to the facet replacement prosthesis. Similarly, linking the disc replacement to the facet replacement may confer the added benefit of reducing (or redistributing) loading of the anchors (pedicle, lamina, spinous process and/or some combination thereof) of the facet replacement prosthesis, or visa versa (attachment of the disc replacement to the facet replacement affects loading of the disc replacement). Moreover, the forces acting on one component of the device (i.e., the artificial disc replacement device) may be balanced and/or negated by various forces acting on another component of the device (i.e., the facet joint replacement device), thus reducing and/or balancing the forces acting on the entire construct and/or its anchoring devices.

In one embodiment, the connection mechanism between the linkage and the artificial disc replacement can further serve to augment the stability and long-term viability of the artificial disc replacement. In this embodiment, the linkage comprises a longitudinally-extending arm which travels along the endplate of the vertebral body, through an opening formed in the artificial disc replacement, and extending further along the endplate. Desirably, this arm will serve to distribute loading of the disc on the endplate, reducing and/or eliminating subsidence of the disc replacement into and/or through the vertebral endplate (in a manner similar to using a rescue ladder on thin ice to distribute the weight of the rescuer). Various embodiments of the arm can comprise a flattened or half-circular cross-section, with the flattened section (towards the endplate) comprising a bioactive and/or in-growth surface to promote biofixation to the surrounding tissues. The linkage arms could comprise flexible or rigid materials.

In one alternate embodiment, the linkage arms are desirably non-parallel and/or non symmetric between the upper and lower linkage arms (which are linked to the upper and lower components of the disc replacement, respectively), so as to provide both lateral and anterior/posterior support to prevent migration of the disc replacement device and/or more easily allow controlled displacement of the disc replacement upon manipulation of the linkage arms.

If desired, a displaceable/repositionable disc replacement system (as described in the paragraph above) could incorporate one or more “settings” that would allow the physician to control, limit, reduce, increase or prevent motion of the disc replacement and/or facet replacement devices (to promote some clinical benefit, including inducing spinal fusion, limit articulation to promote healing of spinal tissues, limit or allow micro motion to promote bony in-growth into devices, or some other desired clinical outcome).

In various embodiments, the linkage between the facet replacement prosthesis and the disc replacement device facilitates positioning (or repositioning) of the respective prosthesis/device relative to each other, to more easily allow matching (or compatibility) of the kinematics and/or performance characteristics of the prosthesis/devices to each other (desirably, to emulate the natural spinal joint).

In various embodiments, the disc replacement device could incorporate openings or other docking features that could be utilized, at a later date (such as, for example, during a subsequent surgical procedure), to attach a facet replacement device (as disclosed herein) to the disc replacement. For example, where the disc replacement has been implanted, and the patient has healed from that surgery, but suffers spinal degeneration in the future (such as, for example, degenerated facets, spinal stenosis and/or spondylolitic slip of the treated spinal level), the level can be reopened, the facet replacement device attached to the existing disc replacement implant, and the surgical procedure completed. A similar arrangement could be contemplated for a facet replacement device that is initially implanted with openings or docking features that are later utilized during subsequent implantation of an artificial disk replacement prosthesis.

Various alternative embodiments of the present invention relate to laminar and/or pedicle based systems for replacing natural facets, the systems anchored to the vertebral bodies, with or without using cement and/or bony ingrowth surfaces to augment fixation.

As will be appreciated by those skilled in the art, the various embodiments disclosed herein can be adapted to account for location, length and orientation of, for example, the laminar passage created by the surgeon during implantation. The various embodiments can also be adapted to account for an individual patient's anatomical constraints. Thus, a limited number of component sizes and/or shapes can be configured from a kit to accommodate a large variety of anatomical variations possible in a patient. For example, a kit including a cephalad implant can include cephalad implants having various lengths from 20 mm to 70 mm, in, for example, 5 or 10 mm increments to accommodate passages/lamina having different lengths/thicknesses. Similarly the depth of apertures that accommodate a component can also be adapted to accommodate a patient.

Another advantage of various embodiments is that the use of the lamina and spinous process as an anchor point for the device enables the device to be implanted while avoiding the pedicles of the vertebral body. Alternatively, it may be desirous to utilize the pedicles of the vertebral body as an anchor point for the device while avoiding the lamina and spinous process. In various embodiments, the combination of translaminar and pedicular attachment (or a hybrid of both) may be most advantageous to the patient. For example, where facet replacement devices are implanted into multiple spinal levels, such as implantation of facet replacement devices across each of the L4-S1 levels, the use of a cephalad translaminar facet replacement device (in the L4 vertebra) in combination with a caudad pedicular-anchored facet replacement device (in the L5 vertebra) may be used in the L4-L5 level, while the use of a cephalad pedical-anchored facet replacement device (in the L5 vertebra—potentially utilizing the same pedicle anchors as for the caudad components of the L4-L5 level) in combination with a caudad pedicular-anchored device (in the sacrum) may be used in the L5-|S1 level. Such an arrangement would thus obviate the need to use the significantly weaker L5 lamina as an anchoring point, yet allow multiple level replacement of the facet joints. Such a hybrid device could, of course, similarly be used in conjunction with all manner of spinal treatment devices, including artificial disc replacements of one or more spinal levels, annular repair, nucleus replacement, dynamic stabilization, ligament repair and replacement, interspinous spacer, articulating rod and screw systems, and/or adjacent level fusion devices.

Additional disclosure useful in understanding the scope and teaching of the invention as it relates to intervertebral discs is in U.S. Patent Pubs. US 2005/0055096 A1 to Serhan et al., for Functional Spinal Unit Prosthetic; and US 2005/0033434 A1 to Berry for Posterior Elements Motion Restoring Device.

Further disclosures useful in understanding the scope and teaching of the invention are included in U.S. Pat. No. 6,610,091, to Mark A. Reiley, for Facet Arthroplasty Devices and Methods; U.S. Publication Nos. US 2005/0283238 A1, to Mark A. Reiley, for Facet Arthroplasty Devices and Methods; US 2005/0234552 A1, to Mark A. Reiley, for Facet Arthroplasty Devices and Methods; US 2005/0267579 A1, to Mark A. Reiley, et al., for Implantable Device For Facet Joint Replacement; US 2006/0009849 A1, to Mark A. Reiley, for Facet Arthroplasty Devices and Methods; US 2006/0009848 A1, to Mark A. Reiley, for Facet Arthroplasty Devices and Methods; US 2006/0009847 A1, to Mark A. Reiley, for Facet Arthroplasty Devices and Methods; US 2004/0006391 A1, to Mark A. Reiley, for Facet Arthroplasty Devices and Methods; US 2004/0111154 A1, to Mark A. Reiley, for Facet Arthroplasty Devices and Methods; US 2004/0049276 A1, to Mark A. Reiley, for Facet Arthroplasty Devices and Methods; US 2005/0251256 A1, to Mark A. Reiley, for Facet Arthroplasty Devices and Methods; US 2004/0049273 A1, to Mark A. Reiley, for Facet Arthroplasty Devices and Methods; US 2004/0049281 A1, to Mark A. Reiley, for Facet Arthroplasty Devices and Methods; US 2004/0049275 A1, to Mark A. Reiley, for Facet Arthroplasty Devices and Methods; U.S. Pat. No. 6,949,123 B2, to Mark A. Reiley, for Facet Arthroplasty Devices and Methods; U.S. Publication Nos. US 2004/0049274 A1, to Mark A. Reiley, for Facet Arthroplasty Devices and Methods; US 2004/0049278 A1, to Mark A. Reiley, for Facet Arthroplasty Devices and Methods; US 2004/0049277 A1, to Mark A. Reiley, for Facet Arthroplasty Devices and Methods; US 2005/0137706 A1, to Mark A. Reiley, for Facet Arthroplasty Devices and Methods; US 2005/0137705 A1, to Mark A. Reiley, for Facet Arthroplasty Devices and Methods; US 2005/0149190 A1, to Mark A. Reiley, for Facet Arthroplasty Devices and Methods; US 2005/0043799 A1, to Mark A. Reiley, for Facet Arthroplasty Devices and Methods; US 2002/0123806 A1, to Mark A. Reiley, for Facet Arthroplasty Devices and Methods; U.S. Pat. No. 6,974,478, to Mark A. Reiley, et al., for Prostheses, Systems, and Methods for Replacement of Natural Facet Joints with Artificial Facet Joint Surfaces; US 2005/0240265 A1, to Mark Kuiper, et al., for Crossbar Spinal Prosthesis Having a Modular Design and Related Implantation Methods; US 2005/0119748 μl, to Mark A. Reiley, et al., for Prostheses, Systems, and Methods for Replacement of Natural Facet Joints with Artificial Facet Joint Surfaces; US 2005/0027361 A1, to Mark A. Reiley for Facet Arthroplasty Devices and Methods; US 2005/0240266 A1, to Mark Kuiper, et al., for Crossbar Spinal Prosthesis Having a Modular Design and Related Implantation Methods; US 2005/0261770 A1, to Mark Kuiper, et al., for Crossbar Spinal Prosthesis Having a Modular Design and Related Implantation Methods; US 2004/0230201 A1, to Hansen Yuan, et al., for Prostheses, Systems, and Methods for Replacement of Natural Facet Joints with Artificial Facet Joint Surfaces; US 2005/0143818 A1, to Hansen Yuan, et al., for Prostheses, Systems, and Methods for Replacement of Natural Facet Joints with Artificial Facet Joint Surfaces; US 2005/0010291 A1, to David Stinson, et al., for Prostheses, Systems, and Methods for Replacement of Natural Facet Joints with Artificial Facet Joint Surfaces; U.S. application Ser. No. 11/275,447 to David Stinson, et al., for Prostheses, Systems, and Methods for Replacement of Natural Facet Joints with Artificial Facet Joint Surfaces; US 2004/030304 A1, to Hansen Yuan, et al., for Prostheses, Systems, and Methods for Replacement of Natural Facet Joints with Artificial Facet Joint Surfaces; US 2005/0131406 A1, to Mark A. Reiley, et al., for Polyaxial Adjustment of Facet Joint Prostheses; US 2005/0240264A1, to Leonard Tokish, et al., for Anti-rotation Fixation Element for Spinal Prostheses; US 2005/0235508 A1, to Teena M. Augostino, et al., for Facet Joint Prostheses Measurement and Implant tools; U.S. application Ser. No. 11/236,323, to Michael J. Funk, For Implantable Orthopedic Device Component Selection Instrument and Methods; U.S. application Ser. No. 11/206,676, to Richard Broman, et al., for Implantable Spinal Device Revision System; US 2006/0041211 A1, to Teena M. Augostino, et al., for Adjacent Level Facet Arthroplasty Devices, Spine Stabilization Systems, and Methods; US 2006/0041311 A1, to Thomas J. McLeer for Devices and Methods for Treating Facet Joints; U.S. application Ser. Nos. 11/140,570, to Thomas J. McLeer, for Methods and Devices for Improved Bonding to Bone; and Ser. No. 11/244,420, to Thomas J. McLeer, for Polymeric Joint Complex and Methods of Use.

While preferred embodiments of the present invention have been shown and described herein, it will be obvious to those killed in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A facet joint restoration device for use in a restoring a facet joint surface comprising:
(a) a cephalad facet joint element comprising (1) a flexible member adapted to engage a first vertebrae and (2) an artificial cephalad joint; and
(b) a caudad facet joint element comprising (1) a connector adapted for fixation to a second vertebrae and (2) an artificial caudad joint adapted to engage the cephalad facet joint.

2. The facet joint restoration device according to claim 1 wherein the flexible member is adapted to engage a lamina of the first vertebrae.

3. The facet joint restoration device according to claim 1 wherein the cephalad facet joint further comprises a plate with an anchoring mechanism adapted to engage a lamina of the first vertebrae.

4. The facet joint restoration device of claim 3 wherein the anchoring mechanism includes anchoring mechanisms selected from the group consisting of teeth, ridges, nubs, serrations, granulations, a stem, a screw and a spike.

5. The facet joint restoration device of claim 2 wherein the cephalad facet joint element further comprises a second anchoring mechanism for securing the cephalad facet joint element to the first vertebrae.

6. The facet joint restoration device according to claim 2 wherein the connector is adapted for fixation to a pedicle of the second vertebrae.

7. The facet joint restoration device according to claim 5 wherein the second anchoring mechanism comprises a bony in-growth surface.

8. The facet joint restoration device according to claim 1 wherein the device replaces tissue removed from the facet joint.

9. The facet joint restoration device according to claim 1 wherein the device is adapted to restore or maintain motion or mobility for the facet joint.

10. The facet restoration device according to claim 1 wherein a surface of one of the cephalad facet joint element or caudad facet joint element is adapted to contour to an opposing mating surface.

11. The facet restoration device according to claim 1 wherein the artificial caudad joint is a caudad cup having a concave surface.

12. The facet restoration device according to claim 1 wherein the flexible member is a flexible cable.

13. The facet restoration device according to claim 12 wherein the flexible cable is surrounded by a tube.

14. The facet restoration device according to claim 12 wherein the flexible cable is adapted to engage a lock.

15. The facet restoration device according to claim 1 further comprising a spring washer adapted to engage a surface of the first vertebrae.

16. The facet restoration device according to claim 1 further comprising a malleable plate adapted to engage a laminar surface to support the cephalad facet joint element during implantation.

17. A facet joint replacement device for use in replacing all or a portion of a natural facet joint between a first vertebrae and a second vertebrae comprising:
(a) a first cephalad facet joint element having a fixation member adapted to engage a lamina or spinous process of the first vertebrae and;
(b) a first caudad facet joint element, the first caudad facet joint element comprising a first caudad connector adapted to fixate to the second vertebral body and an artificial caudad facet surface adapted to engage with the cephalad facet joint element.

18. The facet joint replacement device according to claim 17 wherein the fixation member is a flexible cable.

19. The facet joint replacement device according to claim 17 further comprising a second cephalad facet joint element and a first crossbar adapted to connect the first cephalad facet joint element to the second cephalad facet joint element.

20. The facet joint replacement device of claim 17 further comprising a second caudad facet joint element and a first crossbar adapted to connect the first caudad facet joint element to the second caudad facet joint element.

21. The facet joint replacement device of claim 19 further comprising a second caudad facet joint element and a second crossbar adapted to connect the first caudad facet joint element to the second caudad facet joint element.

22. The facet replacement device according to claim 17 further comprising a laminar clamp.

23. The facet restoration device according to claim 22 wherein the laminar clamp is adapted to engage the first cephalad facet joint element.

24. The facet replacement device according to claim 23 wherein the laminar clamp further comprises teeth for engaging a laminar surface.

25. The facet replacement device according to claim 23 wherein the laminar clamp is further comprised of a first component and a second component adapted to adjustably engage the lamina.

26. The facet restoration device according to claim 23 wherein the first cephalad facet joint element is adapted to extend from the laminar clamp.

27. The facet replacement device according to claim 17 wherein the artificial caudad facet surface comprises a caudad cup.

28. The facet replacement device according to claim 17 wherein the first cephalad facet joint element rotatably engages the fixation member.

29. The facet replacement device according to claim 18 wherein the flexible cable is surrounded by a tube.

30. The facet replacement device according to claim 17 further comprising a malleable plate adapted to engage a laminar surface to support the cephalad facet joint element.

31. A functional spine unit restoration system for use in a functional spine unit at a vertebral level in a spine comprising:
(a) a first and second cephalad facet joint element;
(b) a first and second caudad facet joint element comprising a connector adapted to secure a vertebral body and an artificial caudad joint adapted to engage the cephalad fact joint;
(c) a crossbar adapted to engage the first caudad facet joint element at a first end and the second caudad facet joint element at a second end; and
(d) an artificial intervertebral disc.

32. The functional spine unit restoration system according to claim 31 wherein the anchor is a flexible cable.

33. The functional spine unit restoration system according to claim 31 wherein the cephalad facet joint further comprises a plate with an anchoring mechanism adapted to engage the lamina.

34. The functional spine unit restoration system of claim 33 wherein the anchoring mechanism includes anchoring mechanisms selected from the group consisting of teeth, ridges, nubs, serrations, granulations, a stem, and a spike.

35. The functional spine unit restoration system of claim 33 wherein the plate further comprises a threaded rod adapted and configured to engage a threaded aperture of a bearing.

36. The functional spine unit restoration system according to claim 31 wherein the device is configured from naturally occurring materials adapted to form the device, ceramic, metal, or polymer, or combinations thereof.

37. The functional spine unit restoration system according to claim 31 wherein the device restores the biomechanical operation of the functional spine unit.

38. The functional spine unit restoration system according to claim 31 wherein the device treats degenerating or diseased tissue in the target functional spine unit.

39. The functional spine unit restoration system according to claim 31 wherein the device is adapted to restore or maintain motion or mobility for the target functional spine unit.

40. The functional spine unit restoration system according to claim 31 wherein a surface of one of the cephalad joint or caudad joint is adapted to contour to an opposing mating surface.

41. The functional spine unit restoration system according to claim 31 wherein a surface of one of the cephalad joint or caudad joint is adapted to contour to an opposing mating surface.

42. The functional spine unit restoration system according to claim 32 wherein the flexible cable is adapted to engage a lock.

43. The functional spine unit restoration system according to claim 31 further comprising a laminar clamp.

44. The functional spine unit restoration system according to claim 43 wherein the laminar clamp is adapted to engage the crossbar.

45. The functional spine unit restoration system according to claim 43 wherein the laminar clamp further comprises teeth for engaging a laminar surface.

46. The functional spine unit restoration system according to claim 43 wherein the laminar clamp is further comprised of a first component and a second component adapted to adjustably engage the lamina.

47. The functional spine unit restoration system according to claim 43 wherein the cephalad joints are adapted to extend from the laminar clamp.

48. The functional spine unit restoration system according to claim 43 wherein the laminar clamp is further adapted to engage the crossbar.

49. The functional spine unit restoration system according to claim 43 wherein an orientation of a first cephalad joint to a first caudad joint is different than an orientation of a second cephalad joint to a second caudad joint.

50. The functional spine unit restoration system according to claim 43 wherein the laminar clamp is adjustable along a length parallel to a midline of the spine.

51. The functional spine unit restoration system according to claim 31 wherein the artificial caudad joint is a caudad cup.

52. The functional spine unit restoration system according to claim 31 wherein the artificial cephalad joint rotatably engages the flexible cable.

53. The functional spine unit restoration system according to claim 31 wherein the flexible cable is surrounded by a tube.

54. The functional spine unit restoration system according to claim 31 further comprising a spring washer adapted to engage a surface of a vertebral body.

55. The functional spine unit restoration system according to claim 31 further comprising a malleable plate adapted to engage a laminar surface to support the cephalad joint element during implantation.

56. A kit for restoring a functional spine unit at a vertebral level in a spine comprising:
(a) a first and second cephalad facet joint element;
(b) a first and second caudad facet joint element comprising a connector adapted to secure a vertebral body and an artificial caudad joint adapted to engage the cephalad fact joint;
(c) a crossbar adapted to engage the first caudad facet joint element at a first end and the second caudad facet joint element at a second end; and
(d) an artificial intervertebral disc.
